(19) United States
(12) Patent Application Publication
(10) Pub. No.: US 2010/0021529 A1
(43) Pub. Date: Jan. 28, 2010

(54) STEP-DOWN ESTROGEN REGIMEN FOR WOMEN RECEIVING ESTROGEN THERAPY

(76) Inventors: Matthias Schafer, Berlin (GB); Gerard Nahum, Carrboro, NC (US); Vladimir Hanes, Sleepy Hollow, NY (US); Jan-Peter Ingwersen, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/304,600

(22) PCT Filed: Jun. 13, 2007
(86) PCT No.: PCT/EP2007/005195

§ 371 Date: Aug. 4, 2009

Related U.S. Application Data
(60) Provisional application No. 60/812,986, filed on Jun. 13, 2006.
(60) Provisional application No. 60/908,794, filed on Mar. 29, 2007.

(51) Int. Cl. A61K 31/566; A61P 5/30; A61K 9/70

(52) U.S. Cl. 424/449; 514/182; 514/170; 514/171

(57) ABSTRACT

The present invention relates to methods for continuous treatment of diseases, conditions and/or symptoms associated with deficient endogenous levels of estrogen in a woman already receiving a therapeutically effective dose of an estrogen. More particularly, the present invention provides a safe and efficient step-down regimen for women already receiving estrogen therapy and which are potentially overdosed.

This application claims the benefit of the filing dates of U.S. Provisional Application Ser. No. 60/812, 986 filed Jun. 13, 2006, and U.S. Provisional Application Ser. No. 60/908,794 filed Mar. 29, 2007, which are incorporated by reference herein.

### FIELD OF THE INVENTION

This invention relates to methods for continuous treatment of diseases, conditions and/or symptoms associated with deficient endogenous levels of estrogen in a woman already receiving a therapeutically effective dose of an estrogen. More particularly, the present invention provides a safe and efficient step-down regimen for women already receiving estrogen therapy and which are potentially overdosed.

### BACKGROUND OF THE INVENTION

Estrogen deficiency in the perimenopausal and menopausal woman is manifested by both short-term symptoms and long-term system diseases. Menopause typically occurs in women during middle age and is usually associated with short-term symptoms including hot flushes, mood changes, urogenital changes, such as dryness and atrophy of the vagina, sexual dysfunction, and skin changes. Long-term, estrogen deficiency accelerates the risk of chronic diseases such as osteoporosis and cardiovascular disease.

Hot flushes are the most common and bothersome clinical symptom of menopause, affecting approximately 75% of postmenopausal women. The increase in occurrence of hot flushes is linked with the reduction of estrogen levels that go along with menopause. Menopausal symptoms cause discomfort and distress, ranging from tolerable to, at times, severe enough to affect one's quality of life. Currently, there are more than 40 million menopausal women in the US and almost half of them are over the age of 65. As life expectancy continues to increase, most women will spend one-third of their lifetime in menopause.

“Estrogen Replacement Therapy” has been used for several decades for the treatment of estrogen deficiency and has been established as an effective and safe treatment of moderate to severe vasomotor symptoms associated with menopause. However, one of the risks associated with the administration of estrogens is that women with intact uteri develop endometrial hyperplasia referring to over-stimulation of the lining of the uterus, which is a precursor to endometrial or uterine cancer. The development of endometrial hyperplasia is a significant side-effect of Estrogen Replacement Therapy.

It has been shown that progestins can reduce the development of endometrial hyperplasia induced by estrogen therapy. However, side effects often still occur with progestin co-administration. Thus, it is still desirable to have an estrogen replacement therapy in which potential side effects relating to the therapy are reduced.

In order to minimize any potential risks associated with hormone therapy, regulatory authorities, expert groups and professional societies recommend that patients requiring hormone therapy, either estrogen or combined estrogen/progestin therapy, should be treated with the lowest effective dose possible.

At present, the lowest estrogen dose and regimen that will control vasomotor symptoms are recommended. However, administration of the lowest dose to begin estrogen replacement therapy often does not treat severe vasomotor symptoms. Some investigations suggest that a high dose of estrogen (e.g. 1 mg estradiol orally/day) is necessary as starting dose to treat menopausal symptoms. However, a lower dose of estrogen (e.g. 0.5 mg estradiol orally/day) could be used after the initial therapy, and even a lower dose of estrogen can be administered then as a maintenance dose (e.g. 0.3 mg estradiol orally/day).

Despite the fact that lower doses of estrogen can be used after initial therapy, medical practice still deviates considerably from the recommendation of using the lowest effective dose in the treatment of estrogen deficiency. Recent studies show that in 2005, 90% of women were treated with either high dose (>0.66 mg conjugated equine estrogens oral/day or >1.1 mg estradiol oral/day or >55 μg estradiol transdermal/day) or medium doses (>0.42 to ≦0.66 mg conjugated equine estrogens oral/day or >0.7 to ≦1.1 mg estradiol oral/day or >35 to ≦55 μg estradiol transdermal/day).

From responder analyses of recent clinical studies with different estrogen and/or progestin dosages performed by the applicant, it was concluded that at least 65% of highly symptomatic women could be treated with a lower dose than the current standard estrogen or estrogen/progestin doses without any negative impact on efficacy. In consequence, 58.5% (65% out of the 90% taking currently standard dose or higher) of all hormone therapy patients are today overdosed. At least 40% can even be treated with an estrogen dose as low as about 0.3 mg estradiol orally/day or 14 μg estradiol transdermally per day without any negative impact on efficacy. In consequence, 36% (40% out of the 90% taking currently standard dose or higher) of all hormone therapy patients are today substantially overdosed.

When switching down patients directly from standard or high dose estrogen to a very low dose as described above, e.g. 0.3 mg estradiol orally/day or 14 μg estradiol transdermally per day, intolerable estrogen withdrawal symptoms can be expected. This is due to the fact that very rapid decline of estrogen (e.g. after bilateral oophorectomy or during treatment with gonatropin-releasing hormone agonists) leads to more severe climacteric symptoms than a more gradual decline (as in natural menopause).

Thus, there is a great need for suitable step-down estrogen regimens directed to women receiving high or medium dose estrogen therapy, with the purpose of switching the women down to the lowest effective dose without intolerable withdrawal symptoms.

### SUMMARY OF THE INVENTION

The present invention describes a method for switching down women currently under treatment with standard or high estrogen doses to a lower estrogen dose which still provides relief from symptoms associated with estrogen deficiency and a high patient satisfaction.

Thus, in one aspect the present invention relates to a method for continuous treatment of diseases, conditions or symptoms associated with deficient endogenous levels of estrogen in a woman already receiving a therapeutically effective dose of an estrogen, comprising administration of a first dose of an estrogen wherein the first dose is equal to or less than the therapeutically effective dose of an estrogen already administered to the woman, followed by administration of one or more lower doses of an estrogen, and optionally continuous administration of the lowest dose of the administered estrogen. Another important aspect of the invention is directed to the simultaneous protection of the endometrium from adverse effects of estrogen. This may be achieved by (partly) co-administration of a progestin. Therefore, the invention further relates to a combination treatment comprising administration of an estrogen and a progestin or a selective estrogen receptor modulator (SERM). An example of a progestin according to the invention is drospirenone. Examples of SERMs are e.g. ralozifene or bazedoxifene.

Another aspect of the invention relates to the use of an estrogen for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) optionally continue treatment by administering to said woman, a therapeutically effective amount of an estrogen which is equal to or less than said third therapeutically effective amount of estrogen.

A further aspect of the invention relates to use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a daily oral dose of from 0.9 to 1.1 mg estradiol for 0.5×28 to 2×28 days; and then
(ii) administering to said woman a daily oral dose of from >0.4 to 0.65 mg estradiol for 0.5×28 to 2×28 days; and then;
(iii) administering to said woman a daily oral dose of from 0.2 to 0.4 mg estradiol for 0.5×28 to 2×28 days; and
(iv) optionally continue treatment by administering to said woman, a therapeutically effective amount of estradiol which is equal to or less than the dose specified in (iii).

In yet a further aspect, the invention relates to use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(ii) administering to said woman a daily oral dose of from >0.4 to 0.65 mg estradiol for 1×28 to 2×28 days; and then;
(iii) administering to said woman a daily oral dose of from 0.2 to 0.4 mg estradiol for 1×28 to 2×28 days; and
(iv) optionally continue treatment by administering to said woman, a therapeutically effective amount of estradiol which is equal to or less than the dose specified in (iii).

In still a further aspect, the present invention relates to use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a daily oral dose of from 0.9 to 1.1 mg estradiol for 1×28 to 2×28 days; and then;
(iii) administering to said woman a daily oral dose of from 0.2 to 0.4 mg estradiol for 1×28 to 2×28 days; and
(iv) optionally continue treatment by administering to said woman, a therapeutically effective amount of estradiol which is equal to or less than the dose specified in (iii).

In another aspect, the invention relates to use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a transdermal dose of from 35 to 55 μg estradiol per day for 0.5×28 to 2×28 days; and then
(ii) administering to said woman a transdermal dose of from 20 to 30 μg estradiol per day for 0.5×28 to 2×28 days; and then;
(iii) administering to said woman a transdermal dose of from 12 to 16 μg estradiol per day for 0.5×28 to 2×28 days; and
(iv) optionally continue treatment by administering to said woman, a therapeutically effective amount of estradiol which is equal to or less than the dose specified in (iii).

In yet another aspect, the invention relates to use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(ii) administering to said woman a transdermal dose of from 20 to 30 μg estradiol per day for 1×28 to 2×28 days; and then;
(iii) administering to said woman a transdermal dose of from 12 to 16 μg estradiol per day for 1×28 to 2×28 days; and
(iv) optionally continue treatment by administering to said woman, a therapeutically effective amount of estradiol which is equal to or less than the dose specified in (iii).

In still a further aspect, the invention relates to use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a transdermal dose of from 35 to 55 μg estradiol per day for 1×28 to 2×28 days; and then
(iii) administering to said woman a transdermal dose of from 12 to 16 μg estradiol per day for 1×28 to 2×28 days; and
(iv) optionally continue treatment by administering to said woman, a therapeutically effective amount of estradiol which is equal to or less than the dose specified in (iii).

The invention also relates to a method for the continuous treatment of diseases, conditions or symptoms associated with deficient endogenous levels of estrogen in a woman already receiving a therapeutically effective amount of an estrogen, said method comprising the steps of
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) optionally continue treatment by administering to said woman, a therapeutically effective amount of an estrogen which is equal to or less than said third therapeutically effective amount of estrogen.

Furthermore, the invention relates to a pharmaceutical preparation comprising a number of separately packed and individually removable daily oral dosage units placed into a packaging unit, wherein
(i) from 0.5×28 to 2×28 of said daily oral dosage units comprise from 0.9 to 1.1 mg estradiol; and
(ii) from 0.5×28 to 2×28 of said daily oral dosage units comprise from >0.4 to 0.65 mg estradiol; and
(iii) from 0.5×28 to 2×28 of said daily oral dosage units comprise from 0.2 to 0.4 mg estradiol.

In yet a further aspect, the invention relates to a pharmaceutical preparation comprising a number of separately packed and individually removable daily oral dosage units placed into a packaging unit, wherein
(ii) from 1×28 to 2×28 of said daily oral dosage units comprise from >0.4 to 0.65 mg estradiol; and
(iii) from 1×28 to 2×28 of said daily oral dosage units comprise from 0.2 to 0.4 mg estradiol.

In another aspect, the invention relates to a pharmaceutical preparation comprising a number of separately packed and individually removable daily oral dosage units placed into a packaging unit, wherein
(i) from 1×28 to 2×28 of said daily oral dosage units comprise from 0.9 to 1.1 mg estradiol; and
(iii) from 1×28 to 2×28 of said daily oral dosage units comprise from 0.2 to 0.4 mg estradiol.

In yet another aspect, the invention relates to a pharmaceutical preparation comprising a number of separately packed transdermal patches placed into a packaging unit, wherein
(i) one or more of said patches delivers a dose of from 35 to 55 μg estradiol per day; and
(ii) one or more of said patches delivers a dose of from 20 to 30 μg estradiol per day; and
(iii) one or more of said patches delivers a dose of from 12 to 16 μg estradiol per day.

In still a further aspect, the invention relates to a pharmaceutical preparation comprising a number of separately packed transdermal patches placed into a packaging unit, wherein
(ii) one or more of said patches delivers a dose of from 20 to 30 μg estradiol per day; and
(iii) one or more of said patches delivers a dose of from 12 to 16 μg estradiol per day.

Yet another aspect of the present invention is to provide a pharmaceutical preparation comprising a number of separately packed transdermal patches placed into a packaging unit, wherein
(i) one or more of said patches delivers a dose of from 35 to 55 μg estradiol per day; and
(iii) one or more of said patches delivers a dose of from 12 to 16 μg estradiol per day.

The present invention will be described in more detail in the following.

### DETAILED DESCRIPTION OF THE INVENTION

Definitions

Prior to discussing the present invention in further details, the following terms and conventions will first be defined:

The term “deficient endogenous levels of estrogen” as used herein refers to a condition wherein the serum concentration of estrogen is below 20 pg/ml.

Deficient levels of estrogen can be caused by a variety of reasons, such as e.g. natural menopause, peri-menopause, post-menopause, hypogonadism, hysterectomy, castration and/or primary ovarian failure.

The terms “pre-menopause”, “peri-menopause”, “menopause” and “post-menopause” are used in their conventional meaning, e.g. as defined on page 9 of “The Controversial Climateric”; P. A. van Keep et al Ed., MTP Press (1981). More particularly, the term “menopause” is understood as the last natural (ovary-induced) menstruation. It is a single event and a result of an age-dependent dysfunction of the ovarian follicles. Menopause results from the ovaries decreasing their production of the sex hormones estrogen and progesterone. When the number of follicles falls below a certain threshold (a bleeding threshold), the ovaries can no longer produce mature follicles and sex hormones. The ability to reproduce capability ends with menopause. The peri-menopausal phase begins with the onset of climacteric symptoms when the cycle becomes irregular and ends one year after menopause. The end of peri-menopausal phase can be identified after a protracted period of time without bleeding. Post-menopause is the phase that begins at menopause and continues until death.

“Hypogonadism” in females is a term for a defect of the reproductive system which results in lack of function of the gonads (ovaries). The ovaries have two functions: To produce hormones (e.g estradiol) and to produce gametes (eggs). Deficiency of sex hormones, such as estradiol can result in defective primary or secondary sexual development, or withdrawal effects (e.g., premature menopause) in adults. The term hypogonadism is usually applied to permanent rather than transient or reversible defects, and usually implies deficiency of reproductive hormones such as estradiol, with or without fertility defects.

“Hysterectomy” is the surgical removal of the uterus. A total hysterectomy is removal of the uterus and cervix. A partial hysterectomy is removal of the uterus leaving the stump of the cervix (also called supra-cervical). Hysterectomy can include the surgical removal of the ovaries (oophorectomy). Removal of the female gonads, the ovaries, is female castration. Women who undergo total hysterectomy with bilateral salpingo-oophorectomy (removal of both ovaries, i.e. castration) lose most of their hormone production, including many estrogens and progestins. A woman who is undergoing natural menopause has intact and functional female organs, while a woman who has been hysterectomized and castrated does not. Accordingly, in the present context the term “hysterectomized woman” refers to a woman who has undergone total or completely hysterectomy.

The term “a woman already receiving a therapeutically effective amount of an estrogen”, as used herein, refers to woman who is currently receiving estrogen therapy and who is potentially overdosed, i.e. receiving a medium or a high dose of an estrogen.

The term “high dose of an estrogen”, as used herein, refers to an amount of an estrogen equal to a therapeutically equivalent amount of conjugated equine estrogens (CEE), wherein more than 0.66 mg CEE is administered orally per day, or equal to a therapeutically equivalent amount of estradiol, wherein more than 1.1 mg estradiol is administered orally per day, or equal to a therapeutically equivalent amount of estradiol, wherein more than 55 μg estradiol is administered transdermally per day (cf. Table I further below).

The term “medium dose of an estrogen”, as used herein, refers to an amount of an estrogen equal to a therapeutically equivalent amount of conjugated equine estrogens (CEE), wherein from >0.42 to ≦0.66 mg CEE is administered orally per day, or equal to a therapeutically equivalent amount of estradiol, wherein from >0.7 to ≦1.1 mg estradiol is administered orally per day, or equal to a therapeutically equivalent amount of estradiol, wherein from >35 to ≦55 μg estradiol is administered transdermally per day (cf. Table I further below).

The term “low dose of an estrogen”, as used herein, refers to an amount of an estrogen equal to a therapeutically equivalent amount of conjugated equine estrogens (CEE), wherein from >0.18 to ≦0.42 mg CEE is administered orally per day, or equal to a therapeutically equivalent amount of estradiol, wherein from >0.3 to ≦0.7 mg estradiol is administered orally per day, or equal to a therapeutically equivalent amount of estradiol, wherein from >15 to ≦35 μg estradiol is administered transdermally per day (cf. Table I further below).

The term “very low dose of estrogen”, as used herein, refers to an amount of an estrogen equal to a therapeutically equivalent amount of conjugated equine estrogens (CEE), wherein ≦0.18 mg CEE is administered orally per day, or equal to a therapeutically equivalent amount of estradiol, wherein ≦0.3 mg estradiol is administered orally per day, or equal to a therapeutically equivalent amount of estradiol, wherein ≦15 μg estradiol administered transdermally per day (cf. Table I further below).

The term “therapeutically equivalent amount of conjugated equine estrogens”, means that other estrogens are administered in amounts which give rise to the same therapeutic effect as does the specified amount of conjugated equine estrogens.

The term “continuous treatment”, as used herein, refers to a treatment that follows a previous treatment and which treats or alleviates the diseases, condition and/or symptoms subject to the previous treatment to the same extent as the previous treatment, such as e.g. lessening the frequency, persistence, duration and/or severity of diseases, conditions or symptoms subject to the previous treatment to the same extent as the previous treatment, or such as e.g. results in an effective alleviation of the frequency, persistence, duration and/or severity of diseases, conditions or symptoms subject to the previous treatment to the same extent as the previous treatment, or such as e.g. results in a complete arrest of the frequency, persistence, duration and/or severity of diseases, conditions or symptoms subject to the previous treatment.

The term “equal to the therapeutically effective amount of an estrogen already administered to a woman”, as used herein, refers to the same therapeutically effective amount of an estrogen already administered to the woman or a therapeutically effective amount which is from 90% to 110% of the therapeutically effective amount of an estrogen already administered to the woman, such as 95% to 105% of the therapeutically effective amount of an estrogen already administered to the woman, e.g. 98% to 102% of the therapeutically effective amount of an estrogen already administered to the woman.

In the present context, the term “a first therapeutically effective amount of an estrogen”, when used in connection with continuous treatment of a woman already receiving a therapeutically effective amount of an estrogen, means a therapeutically effective amount of estrogen that is equal to or less than the therapeutically effective amount of estrogen already administered to said woman, and which is sufficient to lessening the frequency, persistence, duration and/or severity of the symptoms associated with estrogen deficiency, such as hot flushes, to the same extent as the therapeutically effective amount of the estrogen which the woman is already receiving. Preferably, the “first therapeutically effective amount” is capable of maintaining the woman subject to the estrogen treatment free of symptoms, such as hot flushes, associated with deficient endogenous levels of estrogen and/or the “first therapeutically effective amount” is capable of preventing relapse of symptoms, such as hot flushes, associated with deficient endogenous levels of estrogen. In some embodiments of the present invention, the term “a first therapeutically effective amount of an estrogen” is also referred to as “a therapeutically effective amount (A) of an estrogen”.

Likewise, the term “a second therapeutically effective amount of an estrogen”, when used in connection with continuous treatment of a woman already receiving a therapeutically effective amount of an estrogen, means a therapeutically effective amount of the estrogen that is less than the first therapeutically effective amount of an estrogen, as defined above, and which is sufficient to lessening the frequency, persistence, duration and/or severity of the symptoms associated with deficient endogenous levels of estrogen, such as hot flushes, to the same extent as the therapeutically effective amount of an estrogen which the woman is already receiving. Preferably, the “second therapeutically effective amount” is capable of maintaining the woman subject to the estrogen treatment free of symptoms, such as hot flushes, associated with deficient endogenous levels of estrogen and/or the “second therapeutically effective amount” is capable of preventing relapse of symptoms, such as hot flushes, associated with deficient endogenous levels of estrogen. In some embodiments of the present invention, the term “a second therapeutically effective amount of an estrogen” is also referred to as “a therapeutically effective amount (B) of an estrogen”.

The term a “third therapeutically effective amount of an estrogen”, when used in connection with continuous treatment of a woman already receiving a therapeutically effective amount of an estrogen, means a therapeutically effective amount of an estrogen that is less than the second therapeutically effective amount of an estrogen, as defined above, and which is sufficient to lessening the frequency, persistence, duration and/or severity of the symptoms, such as hot flushes, associated with deficient endogenous levels of estrogen, to the same extent as the therapeutically effective amount of an estrogen which the woman is already receiving. Preferably, the “third therapeutically effective amount” is capable of maintaining the woman subject to the estrogen treatment free of symptoms, such as hot flushes, associated with deficient endogenous levels of estrogen and/or the “second therapeutically effective amount” is capable of preventing relapse of symptoms, such as hot flushes, associated with deficient endogenous levels of estrogen. In some embodiments of the present invention, the term “a third therapeutically effective amount of an estrogen” is also referred to as “a therapeutically effective amount (C) of an estrogen”.

The term “first treatment period” as used herein, refers to a period of estrogen therapy where the woman is treated continuously, e.g. daily, with a first therapeutically effective amount of an estrogen. The “first treatment period” is continued for 0.5×28 to 6×28 days, such as for 0.5×28 to 5×28 days, or such as for 0.5×28 to 4×28 days, or such as for 0.5×28 to 3×28 days, or such as for 0.5×28 to 2×28 days, or such as for 0.5×28 to 1×28 days. Preferably, the “first treatment period” is continued for 0.5×28 to 4×28 days, more preferably for 0.5×28 to 2×28 days. Thus, the “first treatment period” can be continued for 0.5×28 days, or 1×28 days, or 1.5×28 days, or 2×28 days, or 2.5×28 days, or 3×28 days, or 3.5×28 days, or 4×28 days, or 4.5×28 days, or 5×28 days, or 5.5×28 days, or 6×28 days. Most preferably, the “first treatment period” is continued for 1×28 days or 1.5×28 days. In some embodiments of the present invention, the term “first treatment period” is also referred to as “treatment period (A′)”.

Likewise, the term “second treatment period” as used herein, refers to a period of estrogen therapy where the woman is treated continuously, e.g. daily, with a second therapeutically effective amount of an estrogen. The “second treatment period” is continued for 0.5×28 to 6×28 days, such as for 0.5×28 to 5×28 days, or such as for 0.5×28 to 4×28 days, or such as for 0.5×28 to 3×28 days, or such as for 0.5×28 to 2×28 days, or such as for 0.5×28 to 1×28 days. Preferably, the “second treatment period” is continued for 0.5×28 to 4×28 days, more preferably for 0.5×28 to 2×28 days. Thus, the “second treatment period” can be continued for 0.5×28 days, or 1×28 days, or 1.5×28 days, or 2×28 days, or 2.5×28 days, or 3×28 days, or 3.5×28 days, or 4×28 days, or 4.5×28 days, or 5×28 days, or 5.5×28 days, or 6×28 days. Most preferably, the “second treatment period” is continued for 1×28 days or 1.5×28 days. In some embodiments of the present invention, the term “second treatment period” is also referred to as “treatment period (B′)”.

The term “third treatment period” as used herein, refers to a period of estrogen therapy where the woman is treated continuously, e.g. daily, with a third therapeutically effective amount of an estrogen. The “third treatment period” is continued for 0.5×28 to 6×28 days, such as for 0.5×28 to 5×28 days, or such as for 0.5×28 to 4×28 days, or such as for 0.5×28 to 3×28 days, or such as for 0.5×28 to 2×28 days, or such as for 0.5×28 to 1×28 days. Preferably, the “third treatment period” is continued for 0.5×28 to 4×28 days, more preferably for 0.5×28 to 2×28 days. Thus, the “third treatment period” can be continued for 0.5×28 days, or 1×28 days, or 1.5×28 days, or 2×28 days, or 2.5×28 days, or 3×28 days, or 3.5×28 days, or 4×28 days, or 4.5×28 days, or 5×28 days, or 5.5×28 days, or 6×28 days. Most preferably, the “third treatment period” is continued for 1×28 days or 1.5×28 days. In some embodiments of the present invention, the term “third treatment period” is also referred to as “treatment period (C′)”.

The term “treatment period”, when is used herein, refers to all of the various treatment periods defined above, i.e. to the “first treatment period”, to the “second treatment period” and to the “third treatment period”. Accordingly, when the term “treatment period” is used herein, all statements and details given in that connection apply equally to the first, second and third treatment period.

The term “estrogen” is meant to encompass all compounds (natural or synthetic, steroidal or non-steroidal compounds) exhibiting estrogenic activity. Such compounds encompass natural and synthetic estradiol and its derivatives; conjugated estrogens; estrogen receptor specific agonists; and non-steroidal compounds exhibiting estrogenic activity. The term is further meant to encompass all isomeric and physical forms of the estrogens including hydrates, such as a hemihydrate; solvates; salts; and complexes, such as complexes with cyclodextrins. A preferred estrogen is estradiol and therapeutically acceptable derivatives thereof.

When used herein, the term “therapeutically acceptable derivative of estradiol” refers to esters, such as sulfate esters, of estradiol; salts of estradiol and estradiol esters, such as sodium salts, e.g. sodium salts of sulfate esters; as well as other derivatives known in the art. Typically, an ester of estradiol is in the 3-position or 7-position of estradiol. Specific examples of typical esters of estradiol include estradiol valerate, estradiol acetate, estradiol propionate, estradiol enantate, estradiol undecylate, estradiol benzoate, estradiol cypionate, estradiol sulfate, estradiol sulfamate, as well as salts thereof.

The term “estradiol” is intended to mean that the estradiol may be in the form of 17-α-estradiol or 17-β-estradiol. Preferably, the estradiol is in the form of 17-β-estradiol. The term “estradiol” also covers hydrated forms of estradiol, in particular estradiol hemihydrate.

By the term “conjugated estrogen” is meant the natural conjugated estrogens, such as estrone and equilin and others obtained from pregnant mare urine (as used in Premarin® or Prempro®). Conjugated estrogens are also made synthetically. Examples of synthetically produced estrogens include estropipate and ethinyl estradiol. Further, the term “conjugated estrogens” refers to esters of such compounds, such as the sulfate esters, salts of such compounds, such as sodium salts, and esters of the salts of such compounds, such as sodium salts of a sulfate ester, as well as other derivatives known in the art. Some specific examples include 17-α and β-dihydroequilin, equilenin, 17-α and β-dihydro-equilenin, estrone, and their sodium sulfate esters.

In the present context, the term “progestin” covers synthetic progestagens (also sometimes termed progestogens or gestagens). Thus, the term “progestin” covers hormone compounds exhibiting progestogenic activity and/or which exert anti-estrogenic (counteracting the effects of estrogens in the body) and/or anti-gonadotropic (inhibiting the production of sex steroids and gonads) properties. Progestins are classified according to the structure as C-19 and C-21 progestins, where the C-19 progestins are derived from testosterone and the C-21 progestins are derived from progesterone. Specific examples of progestins include, but is not limited to, progestins selected from the group consisting of levo-norgestrel, dl-norgestrel, norethindrone(norethisterone), norethindrone(norethisterone)acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desorgestrel, norgestimate, gestodene, tibolone, cyproterone acetate, progesterone, and drospirenone. A particular preferred progestin is drospirenone.

The term “SERM” is short for selective estrogen receptor modulator. SERMs act like estrogen in some tissues, while at other times blocking the effects of estrogen. Thus, SERMs are compounds with organ specific estrogen agonistic or estrogen antagonistic activity. SERMs which possess estrogen antagonistic activity at the endometrium are of importance since they can be used instead of a progestin to protect the endometrium from estrogen induced hyperplasia and subsequent cancer. Examples of SERMs with estrogen antagonistic activity are e.g. ralozifene or bazedoxifene.

The term “therapeutically equivalent amount of estradiol”, means that other estrogens, or other forms of estradiol, such as estradiol hemihydrate, are administered in amounts which give rise to the same therapeutic effect as does the specified amount of estradiol. Likewise, the term “therapeutically equivalent amount of drospirenone” means that other progestins are administered in amounts which give rise to the same therapeutic effect as does the specified amount of drospirenone. It is routine for those skilled in the art to determine therapeutically equivalent amounts or dosages of such other estrogens and/or progestins when the effective dose of estradiol and/or drospirenone is known. For example, the paper of Timmer and Geurts provides guidance of how equivalent doses may be determined (see “Bioequivalence assessment of three different estradiol formulations in postmenopausal women in an open, randomized, single-dose, 3-way cross-over” in European Journal of Drug Metabolism and Pharmacokinetics, 24(1):47-53,1999). Moreover, reference is made to EP 1 253 607 which provides a detailed description of therapeutically equivalent amounts of ethinyl estradiol and estradiol on the one hand, and various progestins on the other hand. For further details concerning determination of dose equivalents of various estrogens and progestins, reference is made to “Probleme der Dosisfindung: Sexualhormone” [Problems of Dose-Finding: Sex Hormones]; F. Neumann et al. in “Arzneimittelforschung” (Pharmaceutical Agent Research) 27, 2a, 296-318 (1977), as well as to “Aktuelle Entwicklungen in der hormonalen Kontrazeption” [Current Developments in Hormonal Contraception]; H. Kuhl in Gynakologe” [Gynecologist] 25: 231-240 (1992).

The present invention relates to methods for continuous treatment of diseases, conditions and/or symptoms associated with deficient endogenous levels of estrogen in a woman already receiving a therapeutically effective dose of an estrogen.

Deficient levels of estrogen can occur for a variety of reasons. For example, deficient levels of estrogen may be caused by e.g. natural menopause, peri-menopause, post-menopause, hypogonadism, castration or ovarian failure. Low levels of estrogen, irrespective of the cause, lead to an overall decreased quality of life for women. Symptoms, diseases and conditions range from merely being inconvenient to life threatening.

Transient symptoms, such as vasomotor signs and psychological symptoms are certainly embodied with the realm of the continuous treatment according to the invention. Vasomotor signs comprise but are not limited to hot flushes, sweating attacks such as night sweats, and palpitations. Psychological symptoms of estrogen deficiency comprise, but are not limited to, insomnia and other sleep conditions, poor memory, loss of confidence, mood changes, anxiety, loss of libido, difficulties in concentration, difficulty in making decisions, diminished energy and drive, irritability and crying spells.

The continuous treatment of the aforementioned symptoms can be associated with the peri-menopausal phase of a woman's life or after, sometimes long time after, menopause. It is anticipated that the continuous treatment according to the invention is applicable to these and other transient symptoms during the peri-menopausal phase, menopause, or post-menopausal phase. Moreover, the aforementioned symptoms can be alleviated if the cause of the estrogen deficiency is hypogonadism, castration or primary ovarian failure.

The continuous treatment according to the invention is also applicable to permanent effects of estrogen deficiency that comprise physical changes such as urogenital atrophy, atrophy of the breasts, cardiovascular disease, changes in hair distribution, thickness of hair, changes in skin condition and osteoporosis.

Urogenital atrophy, and conditions associated with it such as vaginal dryness, increase in vaginal pH and subsequent changes in flora, or events which lead to such atrophy, such as decreases in vascularity, fragmentation of elastic fibres, fusion of collagen fibres, or decreases in cell volume, are symptoms thought to be particularly relevant to the continuous treatment according to the invention. Furthermore, the continuous treatment as described herein is thought to be relevant to other urogenital changes associated with estrogen deficiency, decreases in mucus production, changes in cell population, decreases in glycogen production, decreases in growth of lactobacilli or increases in growth of streptococci, staphylococci, or coliform bacilli. Other associated changes that are thought to be preventable by the continuous treatment according to the invention are those that may render the vagina susceptible to injury or infection, such as exudative discharges, vaginitis, and dyspareunia. Furthermore, infections of the urinary tract and incontinence are other common symptoms associated with lowered estrogen levels.

Other embodiments of the invention include the prevention or alleviation of physical changes associated with estrogen deficiency, such as changes in the skin, changes in hair distribution, thickness of hair, atrophy of the breasts, or osteoporosis.

The prevention and management of osteoporosis, most notably post-menopausal osteoporosis, is a particularly interesting embodiment of the invention. Furthermore, bone demineralisation, reduction of bone mass and density, thinning and interruption of trabeculae, and/or consequent increase in bone fractures or bone deformations are thought to be particularly relevant. The prophylactic treatment of osteoporosis is an interesting therapeutic application of the invention.

A particularly interesting embodiment of the invention is directed to lessening the frequency, persistence, duration and/or severity of hot flushes, sweating attacks, palpitations, sleep conditions, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis (including prevention of osteoporosis), most notably hot flushes, sweating attacks, palpitations, sleep conditions, mood changes, nervousness, anxiety, urogenital atrophy, atrophy of the breasts, as well as prevention or management of osteoporosis.

Another interesting embodiment of the invention is directed to continuous treatment of hot flushes, sweating attacks, palpitations, sleep conditions, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis (including prevention of osteoporosis), most notably hot flushes, sweating attacks, palpitations, sleep conditions, mood changes, nervousness, anxiety, urogenital atrophy, atrophy of the breasts, as well as prevention or management of osteoporosis.

As indicated above, one aspect of the present invention relates to the use of an estrogen for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) optionally continue treatment by administering to said woman, a therapeutically effective amount of an estrogen which is equal to or less than said third therapeutically effective amount of estrogen.

A particular treatment period (e.g. the first treatment period) does not necessarily need to be immediately followed by another treatment period (e.g. the second treatment period), i.e. a treatment-free period may be included between the various treatment periods. However, in a preferred embodiment of the invention the treatment is continued in such a way that a subsequent treatment period follows immediately after the preceding treatment period, i.e. it is generally preferred that no treatment-free periods are included between the treatment periods.

Step i)—First Treatment Period

In a preferred embodiment of the invention, the estrogen is administered orally during the first treatment period. Preferably, the estrogen is administered orally and once daily during the first treatment period.

The therapeutically effective amount of estrogen to be administered during the first treatment period will depend on the amount of estrogen that the woman subject to the continuous treatment of the invention is already receiving. The first therapeutically effective amount is equal to or less than the therapeutically effective amount of the estrogen already administered to said woman.

The first therapeutically effective amount of estrogen according to the invention is sufficient to lessening the frequency, persistence, duration and/or severity of the symptoms associated with estrogen deficiency, such as hot flushes, to the same extent as the therapeutically effective amount of the estrogen which the woman is already receiving.

Preferably, the “first therapeutically effective amount” is capable of maintaining the woman subject to the estrogen treatment free of symptoms, such as hot flushes, associated with deficient endogenous levels of estrogen and/or the “first therapeutically effective amount” is capable of preventing relapse of symptoms, such as hot flushes, associated with deficient endogenous levels of estrogen

In general, however, the amount of estrogen to be administered once daily during the first treatment period typically corresponds to a therapeutically equivalent amount of estradiol of from >0.75 to 1.5 mg per day, preferably in the range of from >0.75 to 1.25 mg per day, more preferably in the range of from 0.9 to 1.1 mg per day, most preferably about 1 mg per day.

While the administered amount of estrogen may be varied within the ranges specified above during the first treatment period, it will be understood that the administered amount of estrogen is preferably the same throughout the first treatment period.

The first treatment period is typically continued for the period of time indicated previously in connection with the definition of the term “first treatment period”.

In another preferred embodiment of the invention, the estrogen is administered transdermally during the first treatment period. Transdermal administration of estrogens by means of patches is known in connection with treatment of estrogen deficiencies. Accordingly, the estrogen to be administered transdermally during the first treatment period may be formulated in any transdermal delivery system known in the art, which is capable of providing the desired release of the estrogen. One example of a commercially available estrogen-containing transdermal delivery system is the Menostar® patch marketed by Berlex, USA.

If the estrogen is administered transdermally, the amount of estrogen to be administered during the first treatment period typically corresponds to a therapeutically equivalent amount of estradiol of from >32.5 to 75 μg per day, preferably in the range of from >32.5 to 62.5 μg per day, more preferably in the range of from 35 to 55 μg per day, most preferably about 37.5 μg per day or about 50 μg per day.

The duration of the first treatment period is the same as described above in connection with oral administration of the estrogen.

In one embodiment of the invention, step (i) is omitted. Thus, according to this embodiment, the invention relates to the use of an estrogen for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(ii) administering to said woman a therapeutically effective amount (B) of an estrogen, also defined herein as the second therapeutically effective amount of an estrogen, during a treatment period (B′), also defined herein as the second treatment period, where said therapeutically effective amount (B) of an estrogen is less than said therapeutically effective amount of an estrogen already administered to said woman; and
(iii) after completion of the treatment period (B′), administering to said woman a therapeutically effective amount (C) of an estrogen, also defined herein as the third therapeutically effective amount of an estrogen, during a treatment period (C′), also defined herein as the third treatment period, where said therapeutically effective amount (C) of an estrogen is less than said therapeutically effective amount (B) of an estrogen; and
(iv) optionally continue treatment by administering to said woman, a therapeutically effective amount of an estrogen which is equal to or less than said therapeutically effective amount (C) of an estrogen.

Step) ii)—Second Treatment Period

In a preferred embodiment of the invention, the estrogen is administered orally during the second treatment period. Preferably, the estrogen is administered orally and once daily during the second treatment period.

The second therapeutically effective amount of estrogen to be administered during the second treatment period is less than the first therapeutically effective amount of the estrogen already administered to said woman.

In a similar way as described above in connection with the first treatment period, the second therapeutically effective amount of estrogen according to the invention is sufficient to lessening the frequency, persistence, duration and/or severity of the symptoms associated with estrogen deficiency, such as hot flushes, to the same extent as the therapeutically effective amount of the estrogen which the woman is already receiving. Preferably, the “second therapeutically effective amount” is capable of maintaining the woman subject to the estrogen treatment free of symptoms, such as hot flushes, associated with deficient endogenous levels of estrogen and/or the “second therapeutically effective amount” is capable of preventing relapse of symptoms, such as hot flushes, associated with deficient endogenous levels of estrogen.

In general, however, the amount of estrogen to be administered once daily during the second treatment period typically corresponds to a therapeutically equivalent amount of estradiol of from >0.4 to 0.75 mg per day, preferably in the range of from >0.4 to 0.65 mg per day, more preferably in the range of from >0.4 to 0.55 mg per day, even more preferably in the range of from 0.45 to 0.55 mg per day, most preferably about 0.5 mg per day.

While the administered amount of estrogen may be varied within the ranges specified above during the second treatment period, it will be understood that the administered amount of estrogen is preferably the same throughout the second treatment period.

The second treatment period is typically continued for the period of time indicated previously in connection with the definition of the term “second treatment period”.

In another preferred embodiment of the invention, the estrogen is administered transdermally during the second treatment period. If the estrogen is administered transdermally, the amount of estrogen to be administered during the second treatment period typically corresponds to a therapeutically equivalent amount of estradiol of from >20 to 32.5 μg per day, preferably in the range of from >20 to 30 μg per day, more preferably in the range of from 22.5 to 27.5 μg per day, most preferably about 25 μg per day.

The duration of the second treatment period is the same as described above in connection with oral administration of the estrogen.

In one embodiment of the invention step (ii), as described above, is omitted. Thus, according to this embodiment, the invention relates to the use of an estrogen for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a therapeutically effective amount (A) of an estrogen, also defined herein as the first therapeutically effective amount of an estrogen, during a treatment period (A′), also defined herein as the first treatment period, where said therapeutically effective amount (A) of an estrogen is equal to or less than the therapeutically effective amount of an estrogen already administered to said woman; and
(iii) after completion of the treatment period (A′), administering to said woman a therapeutically effective amount (C) of an estrogen, also defined herein as the third therapeutically effective amount of an estrogen, during a treatment period (C′), also defined herein as the third treatment period, where said therapeutically effective amount (C) of an estrogen is less than said therapeutically effective amount (A) of an estrogen; and
(iv) optionally continue treatment by administering to said woman, a therapeutically effective amount of an estrogen which is equal to or less than said therapeutically effective amount (C) of an estrogen.

Step) iii)—Third Treatment Period

In a preferred embodiment of the invention, the estrogen is administered orally during the third treatment period. Preferably, the estrogen is administered orally and once daily during the second treatment period.

The third therapeutically effective amount of estrogen to be administered during the third treatment period is less than the second therapeutically effective amount of the estrogen already administered to said woman.

In a similar way as described above in connection with the first and second treatment periods, the third therapeutically effective amount of estrogen according to the invention is sufficient to lessening the frequency, persistence, duration and/or severity of the symptoms associated with estrogen deficiency, such as hot flushes, to the same extent as the therapeutically effective amount of the estrogen which the woman is already receiving. Preferably, the “third therapeutically effective amount” is capable of maintaining the woman subject to the estrogen treatment free of symptoms, such as hot flushes, associated with deficient endogenous levels of estrogen and/or the “third therapeutically effective amount” is capable of preventing relapse of symptoms, such as hot flushes, associated with deficient endogenous levels of estrogen.

In general, however, the amount of estrogen to be administered once daily during the third treatment period typically corresponds to a therapeutically equivalent amount of estradiol of from 0.05 to 0.4 mg per day, preferably in the range of from 0.1 to 0.4 mg per day, more preferably in the range of from 0.2 to 0.4 mg per day, even more preferably in the range of from 0.25 to 0.35 mg per day, most preferably about 0.3 mg per day.

While the administered amount of estrogen may be varied within the ranges specified above during the third treatment period, it will be understood that the administered amount of estrogen is preferably the same throughout the third treatment period.

The third treatment period is typically continued for the period of time indicated previously in connection with the definition of the term “third treatment period”.

In another embodiment of the invention, the estrogen is administered transdermally during the third treatment period. If the estrogen is administered transdermally, the amount of estrogen to be administered during the third treatment period typically corresponds to a therapeutically equivalent amount of estradiol of from 2.5 to 20 μg per day, preferably in the range of from 5 to 20 μg per day, more preferably in the range of from 10 to 20 μg per day, even more preferably in the range of from 12 to 16 μg per day, most preferably about 14 μg per day.

The duration of the third treatment period is the same as described above in connection with oral administration of the estrogen.

Step) iv)

In a specific embodiment, the administration pattern of the medicament of the invention comprises a step (iv), wherein the treatment of the woman already receiving a therapeutically effective amount of an estrogen is continued after the third treatment period by administering to said woman a therapeutically effective amount of an estrogen, which is equal to or less than the third therapeutically effective amount of an estrogen, as defined above.

Thus, in one embodiment the invention relates to the use of an estrogen for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) continue treatment by administering to said woman, a therapeutically effective amount of an estrogen which is equal to or less than said third therapeutically effective amount of estrogen.

In another embodiment of the invention step (iv) is omitted. Thus, according to this embodiment, the invention also relates to the use of an estrogen for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen.

As outlined above, “estrogen” according to the invention, is meant to encompass all compounds (natural or synthetic, steroidal or non-steroidal compounds) exhibiting estrogenic activity. In a preferred embodiment of the invention, the estrogen is estradiol or a salt, hydrate or a therapeutically acceptable derivative thereof. The estradiol may be in the form of 17-α-estradiol or 17-β-estradiol. Preferably, the estradiol is in the form of 17-β-estradiol. The term “estradiol” also covers hydrated forms of estradiol, in particular estradiol hemihydrate. In a more preferred embodiment, the estrogen is estradiol hemihydrate.

In one specific embodiment, the invention relates to the use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a daily oral dose of from 0.9 to 1.1 mg estradiol for 0.5×28 to 2×28 days; and then
(ii) administering to said woman a daily oral dose of from >0.4 to 0.65 mg estradiol for 0.5×28 to 2×28 days; and then;
(iii) administering to said woman a daily oral dose of from 0.2 to 0.4 mg estradiol for 0.5×28 to 2×28 days; and
(iv) optionally continue treatment by administering to said woman, a therapeutically effective amount of estradiol which is equal to or less than the dose specified in (iii).

In a preferred embodiment, the daily oral dose of specified in (i) above is from 0.9 to 1.05 mg estradiol, or from 0.9 to 1.0 mg estradiol, or from 0.95 to 1.1 mg estradiol, or from 0.95 to 1.05 mg estradiol, or about 0.9 mg estradiol, or about 0.95 mg, or about 1 mg, or about 1.05 mg, or about 1.1 mg estradiol, preferably about 1 mg estradiol. In yet a preferred embodiment the daily oral dose of estradiol specified in (i) above is administered for 0.5×28 to 1.5×28 days, or 0.5×28 to 1×28 days, or about 0.5×28 days, or about 1×28 days, or about 1.5×28 days, or about 2×28 days, preferably for about 1×28 days.

In another preferred embodiment, the daily oral dose specified in (ii) above is from >0.4 to 0.6 mg estradiol, or from 0.45 to 0.6 mg estradiol, or from 0.5 to 0.6 mg estradiol, or from 0.5 to 0.55 mg estradiol, or about 0.45 mg estradiol, or about 0.5 mg, or about 0.55 mg, or about 0.6 mg, or about 0.65 mg estradiol, preferably about 0.5 mg estradiol. In yet another preferred embodiment, the daily oral dose of estradiol specified in (ii) above is administered for 0.5×28 to 1.5×28 days, or 0.5×28 to 1×28 days, or about 0.5×28 days, or about 1×28 days, or about 1.5×28 days, or about 2×28 days, preferably for about 1×28 days.

In still another preferred embodiment, the daily oral dose of specified in (iii) above is from 0.2 to 0.35 mg estradiol, or from 0.25 to 0.4 mg estradiol, or from 0.25 to 0.35 mg estradiol, or about 0.2 mg estradiol, or about 0.25 mg, or about 0.3 mg, or about 0.35 mg, or about 0.4 mg estradiol, preferably about 0.3 mg estradiol. In a further preferred embodiment, the daily oral dose of estradiol specified in (iii) above is administered for 0.5×28 to 1.5×28 days, or for 1×28 to 2×28 days, preferably for about 1×28 days.

In second specific embodiment, the invention relates to the use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(ii) administering to said woman a daily oral dose of from >0.4 to 0.65 mg estradiol for 1×28 to 2×28 days; and then;
(iii) administering to said woman a daily oral dose of from 0.2 to 0.4 mg estradiol for 1×28 to 2×28 days; and
(iv) optionally continue treatment by administering to said woman, a therapeutically effective amount of estradiol which is equal to or less than the dose specified in (iii).

In a preferred embodiment, the daily oral dose specified in (ii) above is from >0.4 to 0.6 mg estradiol, or from >0.4 to 0.55 mg estradiol, or from >0.4 to 0.5 mg estradiol, or from >0.4 to 0.45 mg estradiol, or from 0.45 to 0.65 mg estradiol, or from 0.45 to 0.6 mg estradiol, or from 0.45 to 0.55 mg estradiol, or about 0.45 mg, or about 0.5 mg, or about 0.6 mg, or about 0.65 mg estradiol, preferably about 0.5 mg estradiol. In yet a specific embodiment, the daily oral dose specified in (ii) above is administered for 1×28 to 1.5×28 days, or for 1.5×28 to 2×28 days, or for about 1×28 days, or about 1.5×28 days or about 2×28 days. In another specific embodiment, the daily oral dose specified in (ii) is administered for about 1.5×28 days.

In another preferred embodiment, the daily oral dose of the medicament according to the invention specified in (iii) above is from 0.2 to 0.35 mg estradiol, or from 0.25 to 0.4 mg estradiol, or from 0.25 to 0.35 mg estradiol, or about 0.2 mg estradiol, or about 0.25 mg, or about 0.3 mg, or about 0.35 mg, or about 0.4 mg, preferably about 0.3 mg estradiol.

In yet a preferred embodiment, the daily oral dose specified in (iii) above is administered for 1×28 to 1.5×28 days, or for 1.5×28 to 2×28 days, or for about 1×28 days, or for about 1.5×28 days, or for about 2×28 days, preferably for about 1.5×28 days.

In a third specific embodiment, the invention relates to the use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a daily oral dose of from 0.9 to 1.1 mg estradiol for 1×28 to 2×28 days; and then;
(iii) administering to said woman a daily oral dose of from 0.2 to 0.4 mg estradiol for 1×28 to 2×28 days; and
(iv) optionally continue treatment by administering to said woman, a therapeutically effective amount of estradiol which is equal to or less than the dose specified in (iii).

In a preferred embodiment, the daily oral dose specified in (i) above is from 0.9 to 1.05 mg estradiol, or from 0.9 to 1.0 mg estradiol, or from 0.95 to 1.1 mg estradiol, or from 0.95 to 1.05 mg estradiol, or about 0.9 mg estradiol, or about 0.95 mg, or about 1 mg, or about 1.05 mg, or about 1.1 mg estradiol, preferably about 1 mg estradiol. In yet a preferred embodiment the daily oral dose of estradiol specified in (i) above is administered for 1×28 to 1.5×28 days, or for 1.5×28 to 2×28 days, or for about 1×28 days, or for about 1.5×28 days, or for about 2×28 days, preferably for about 1.5×28 days.

In another preferred embodiment, the daily oral dose of specified in (iii) above is from 0.2 to 0.35 mg estradiol, or from 0.25 to 0.4 mg estradiol, or from 0.25 to 0.35 mg estradiol, or about 0.2 mg estradiol, or about 0.25 mg, or about 0.3 mg, or about 0.35 mg, or about 0.4 mg, preferably about 0.3 mg estradiol.

In yet a preferred embodiment, the daily oral dose specified in (iii) above is administered for 1×28 to 1.5×28 days, or for 1.5×28 to 2×28 days, or for about 1×28 days, or for about 1.5×28 days, or for about 2×28 days, preferably for about 1.5×28 days.

In a fourth specific embodiment, the invention relates to the use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a transdermal dose of from 35 to 55 μg estradiol per day for 0.5×28 to 2×28 days; and then
(ii) administering to said woman a transdermal dose of from 20 to 30 μg estradiol per day for 0.5×28 to 2×28 days; and then;
(iii) administering to said woman a transdermal dose of from 12 to 16 μg estradiol per day for 0.5×28 to 2×28 days; and
(iv) optionally continue treatment by administering to said woman, a therapeutically effective amount of estradiol which is equal to or less than the dose specified in (iii).

In a preferred embodiment the daily transdermal dose specified in (i) above is from 35 to 50 μg estradiol, or from 35 to 45 μg estradiol, or from 35 to 40 μg estradiol, or from 40 to 55 μg estradiol, or from 40 to 50 μg estradiol, or from 40 to 45 μg estradiol, or from 45 to 55 μg estradiol, or from 45 to 50, or from 50 to 55 μg estradiol, preferably 45 to 55 μg 5 estradiol. In another preferred embodiment, the daily transdermal dose specified in (i) above is about 35 μg estradiol, or about 40 μg estradiol, or about 45 μg estradiol, or about 50 μg estradiol, or about 55 μg estradiol, preferably about 37.5 μg or about 50 μg estradiol.

In yet another preferred embodiment, the daily transdermal dose specified in (i) above is administered for 0.5×28 to 1.5×28 days, or for 0.5×28 to 1×28 days, or for 1×28 to 2×28 days, or for 1×28 to 1.5×28 days, preferably for about 1×28 days.

In a further preferred embodiment, the daily transdermal dose specified in (ii) above is from 20 to 25 μg estradiol, or from 25 to 30 μg estradiol, or about 20 μg estradiol, or about 22.5 μg estradiol, or about 25 μg estradiol, or about 27.5 μg estradiol, or about 30 μg estradiol, preferably about 25 μg estradiol.

In yet a further embodiment, the daily transdermal dose specified in (ii) above is administered for 0.5×28 days to 1.5×28 days, or for 1×28 to 2×28 days, or for 1×28 to 1.5×28 days, preferably for about 1×28 days.

In another embodiment, the daily transdermal dose specified in (iii) above is from 12 to 15 μg estradiol, or from 12 to 14 μg estradiol, or from 12 to 13 μg estradiol, or from 13 to 16 μg estradiol, or from 13 to 15 μg estradiol, or from 13 to 14 μg estradiol, or from 14 to 16 μg estradiol, or from 14 to 15 μg estradiol, or from 15 to 16 μg estradiol, preferably from 13 to 15 μg estradiol. In a further embodiment, the daily transdermal dose specified in (iii) above is about 12 μg estradiol, or about 13 μg estradiol, or about 14 μg estradiol, or about 15 μg estradiol, or about 16 μg estradiol.

The daily transdermal dose specified in (iii) above can be administered for 0.5×28 to 1.5×28 days, or for 0.5×28 to 1×28 days, or for 1×28 to 2×28 days, or for 1×28 to 1.5×28 days, or for 1.5×28 to 2×28 days, preferably for 0.5×28 to 1.5×28 days. The daily transdermal dose specified in (iii) above can also be administered for about 0.5×28 days, or for about 1×28 days, or for about 1.5×28 days, or for about 2×28 days, preferably for about 1×28 days.

In a fifth specific embodiment, the invention relates to the use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(ii) administering to said woman a transdermal dose of from 20 to 30 μg estradiol per day for 1×28 to 2×28 days; and then;
(iii) administering to said woman a transdermal dose of from 12 to 16 μg estradiol per day for 1×28 to 2×28 days; and
(iv) optionally continue treatment by administering to said woman, a therapeutically effective amount of estradiol which is equal to or less than the dose specified in (iii).

The daily transdermal dose specified in (ii) is from 20 to 25 μg estradiol, or from 25 to 30 μg estradiol, or about 20 μg estradiol, or about 22.5 μg estradiol, or about 25 μg estradiol, or about 27.5 μg estradiol, or about 30 μg estradiol, preferably about 25 μg estradiol per day.

In yet a further embodiment, the daily transdermal dose specified in (ii) is administered for 0.5×28 days to 1.5×28 days, or for 1×28 to 2×28 days, or for 1×28 to 1.5×28 days, preferably for about 1.5×28 days.

In another embodiment, the daily transdermal dose specified in (iii) is from 12 to 15 μg estradiol, or from 12 to 14 μg estradiol, or from 12 to 13 μg estradiol, or from 13 to 16 μg estradiol, or from 13 to 15 μg estradiol, or from 13 to 14 μg estradiol, or from 14 to 16 μg estradiol, or from 14 to 15 μg estradiol, or from 15 to 16 μg estradiol, preferably from 13 to 15 μg estradiol. In a further embodiment, the daily transdermal dose specified in (iii) is about 12 μg estradiol, or about 13 μg estradiol, or about 14 μg estradiol, or about 15 μg estradiol, or about 16 μg estradiol, preferably about 14 μg estradiol per day.

The daily transdermal dose specified in (iii) can be administered for 0.5×28 to 1.5×28 days, or for 0.5×28 to 1×28 days, or for 1×28 to 2×28 days, or for 1×28 to 1.5×28 days, or for 1.5×28 to 2×28 days, preferably for 0.5×28 to 1.5×28 days. The daily dose specified in (iii) can also be administered for about 0.5×28 days, or for about 1×28 days, or for about 1.5×28 days, or for about 2×28 days, preferably for about 1.5×28 days.

In a sixth specific embodiment, the invention relates to the use of estradiol for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a transdermal dose of from 35 to 55 μg estradiol per day for 1×28 to 2×28 days; and then
(iii) administering to said woman a transdermal dose of from 12 to 16 μg estradiol per day for 1×28 to 2×28 days; and
(iv) optionally continue treatment by administering to said woman, a therapeutically effective amount of estradiol which is equal to or less than the dose specified in (iii).

In a preferred embodiment the daily transdermal dose specified in (i) is from 35 to 50 μg estradiol, or from 35 to 45 μg estradiol, or from 35 to 40 μg estradiol, or from 40 to 55 μg estradiol, or from 40 to 50 μg estradiol, or from 40 to 45 μg estradiol, or from 45 to 55 μg estradiol, or from 45 to 50, or from 50 to 55 μg estradiol, preferably 45 to 55 μg estradiol. In another preferred embodiment, the daily transdermal dose specified in (i) is about 35 μg estradiol, or about 40 μg estradiol, or about 45 μg estradiol, or about 50 μg estradiol, or about 55 μg estradiol, preferably about 37.5 μg or about 50 μg estradiol per day.

In yet another preferred embodiment, the daily transdermal dose specified in (i) is administered for 0.5×28 to 1.5×28 days, or for 0.5×28 to 1×28 days, or for 1×28 to 2×28 days, or for 1×28 to 1.5×28 days, preferably for about 1.5×28 days.

In another embodiment, the daily transdermal dose specified in (iii) is from 12 to 15 μg estradiol, or from 12 to 14 μg estradiol, or from 12 to 13 μg estradiol, or from 13 to 16 μg estradiol, or from 13 to 15 μg estradiol, or from 13 to 14 μg estradiol, or from 14 to 16 μg estradiol, or from 14 to 15 μg estradiol, or from 15 to 16 μg estradiol, preferably from 13 to 15 μg estradiol. In a further embodiment, the daily transdermal dose specified in (iii) is about 12 μg estradiol, or about 13 μg estradiol, or about 14 μg estradiol, or about 15 μg estradiol, or about 16 μg estradiol, preferably about 14 μg estradiol per day.

The daily transdermal dose specified in (iii) can be administered for 0.5×28 to 1.5×28 days, or for 0.5×28 to 1×28 days, or for 1×28 to 2×28 days, or for 1×28 to 1.5×28 days, or for 1.5×28 to 2×28 days, preferably for 0.5×28 to 1.5×28 days. The daily transdermal dose specified in (iii) can also be administered for about 0.5×28 days, or for about 1×28 days, or for about 1.5×28 days, or for about 2×28 days, preferably for about 1×28 days.

The woman subject to the continuous treatment of the present invention is a woman already receiving a therapeutically effective amount of an estrogen. The estrogen that the woman is already receiving can be any estrogen commonly used in hormone therapies known in the art. When commencing the continuous treatment of the invention, the woman normally discontinues the previous treatment. Thus, in one embodiment the woman who already receives a therapeutically effective amount of one estrogen switches to another estrogen when the first treatment period is initiated. In another embodiment, the woman who already receives a therapeutically effective amount of an estrogen continues treatment with the same estrogen when the first treatment period is initiated, but now receives this estrogen in a lower dose than previously. In yet another embodiment, the woman who already receives a therapeutically effective amount of an estrogen continues treatment with the same estrogen when the first treatment period is initiated and receives this estrogen in the same dose as previously. As will be understood, in the later case the woman must then necessarily receive a lower dose of the estrogen when the second treatment period commences.

In one embodiment of the invention, the continuous treatment is directed to a woman that is potentially overdosed, i.e. receiving a hormone therapy comprising a medium or a high dose of an estrogen. According to analyses of the applicant at least 65% of women with menopausal symptoms could be treated with a lower than the current standard dose of estrogen without any negative impact on efficacy and at least 40% can even be treated with an estrogen dose as low as about 0.3 mg estradiol oral per day or 14 μg estradiol transdermal per day without any negative impact on efficacy. In a second embodiment, the continuous treatment of the invention is directed to a woman that is overdosed, i.e. the woman can be treated with an estrogen dose that is lower than a medium or a high dose of estrogen, e.g an estrogen dose about 0.3 mg estradiol oral per day or 14 μg estradiol transdermal per day without any negative impact on efficacy.

The terms medium and high dose of an estrogen are defined above. Table I below outlines the dosage categories high, medium, low and very low of an estrogen. The dosage intervals are defined by dosages of conjugated equine estrogens (CEEs) administered orally, estradiol administered orally and estradiol administered transdermally.

The dosage category of a specific estrogen, which a woman is already receiving is determined by comparing the administered amount of that estrogen with the therapeutically equivalent amounts of CEE or estradiol.

TABLE I

| | Conjugated equine | Estradiol | Estradiol |
|---|---|---|---|
| Dosage | estrogen (oral adm.) | (oral adm.) | (transdermal adm.) |
| categories | (mg/day) | (mg/day) | (μg/day) |
| High dose | >0.66 | >1.1 | >55 |
| Medium dose | >0.42 to ≦0.66 | >0.7 to ≦1.1 | >35 to ≦55 |
| Low dose | >0.18 to ≦0.42 | >0.3 to ≦0.7 | >15 to ≦35 |
| Very low | ≦18 | ≦0.3 | ≦15 |

The woman subject to the continuous treatment according to the invention is already 5 receiving estrogen hormone therapy due to that the woman is suffering from endogenous estrogen deficiency. The estrogen therapy that the woman is already receiving either treats or alleviates the diseases, conditions and/or symptoms associated with endogenous estrogen deficiency, such as e.g. results in a complete arrest of the frequency, persistence, duration and/or severity of diseases, conditions or symptoms associated with endogenous estrogen deficiency or lessens the frequency, persistence, duration and/or severity of diseases, conditions or symptoms associated with endogenous estrogen deficiency.

Thus in one aspect of the invention, the continuous treatment according to the invention is directed to a woman already receiving a therapeutically effective amount of an estrogen due to that said woman is suffering from deficient endogenous levels of estrogen. The woman can either be symptom free or can suffer from diseases, conditions and/or symptoms associated with endogenous estrogen deficiency dependent on the efficacy of the estrogen therapy that the woman is already receiving.

Deficient levels of estrogen can be caused by a variety of reasons, such as e.g. natural menopause, peri-menopause, post-menopause, hypogonadism, hysterectomy, castration, oophorectomy and/or ovarian failure. Thus in one embodiment, the woman subject to the continuous treatment according to the invention is a post-menopausal woman. In a second embodiment, the woman subject to the continuous treatment according to the invention is a hysterectomised woman. In a third embodiment, the woman subject to the continuous treatment according to the invention is suffering from hypogonadism, such as e.g. primary or secondary hypogonadism. In a fourth embodiment, the woman subject to the continuous treatment according to the invention is castrated and/or oophorectomised. In a fifth embodiment, the woman subject to the continuous treatment according to the invention is suffering from ovarian failure, such as e.g. premature ovarian failure.

Combination with Progestin

It is well-established that exogenous estrogens stimulate the proliferation of the endometrium. In estrogen monotherapy, the opposing effect of progesterone, which terminates proliferation, is absent. The desquamation phase, during which the top layers of the endometrium are shed, does not occur and proliferation of the endometrium occurs to a greater extent than in the phases up to and including the pre-menopausal phase. The result is hyperplasia, a risk factor for endometrial cancer. Combination therapy, also referred to as opposed therapy, is a treatment where a progestin is added to protect the endometrium from hyperplasia. Accordingly, in a preferred embodiment of the invention, in particular in connection with continuous treatment of diseases, conditions or symptoms associated with deficient endogenous levels of estrogen in a woman already receiving a therapeutically effective amount of an estrogen and who has not undergone hysterectomy (a “non-hysterectomized woman”), co-administration of a progestin for one or more sub-periods of the treatment periods is desirable in order to protect the endometrium from adverse effects of caused by the exogenous estrogen.

Thus, in one aspect, the present invention relates to the use of a combination of an estrogen and a progestin for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) optionally continue treatment by administering to said woman, a therapeutically effective amount of an estrogen which is equal to or less than said third therapeutically effective amount of estrogen;
   wherein a progestin is administered during the entire treatment period or during a part of the treatment period.

In one embodiment, the progestin is administered during one or more sub-periods of the first treatment period according to the invention and/or during one or more sub-periods of said second treatment period according to the invention and/or during one or more sub-periods of said third treatment period according to the invention and/or during one or more sub-periods of the continuous treatment defined in step (iv).

The progestin can be selected from the group consisting of levo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desorgestrel, norgestimate, gestodene, tibolone, cyproterone acetate and drospirenone. In a preferred embodiment, the progestin is drospirenone.

As will be understood, all statements made above in connection with the aspect concerning administration of the estrogen also apply to the aspect concerning co-administration of a progestin. Thus, all statements made above in connection with the duration of the various treatment periods, the amount of estrogen to be administered in the various treatment periods, ways of administering the estrogen, preferred estrogens to be administered, etc. apply mutatis mutandis to the aspect concerning co-administration of a progestin.

While it is contemplated to administer the progestin via the transdermal route, it is currently preferred that the progestin is administered orally. Accordingly, in one embodiment of the invention, the estrogen is administered transdermally in the various treatment periods while the progestin is administered orally. However, in a preferred embodiment of the invention the estrogen as well as the progestin are administered orally during the various treatment periods. In another embodiment of the invention, the estrogen as well as the progestin are administered transdermally. As will be discussed in more detail infra the estrogen and the progestin may be administered individually, i.e. in individual dosage units. However, in a preferred embodiment of the invention, the estrogen and the progestin are present in same dosage unit and hence administered simultaneously.

When administered orally, the progestin is preferably administered once daily during the one or more sub-periods where the progestin is actually administered. As will be understood, the progestin is typically only administered for one or more, relatively short, sub-periods of the various treatment periods. Thus, during a specific treatment period, i.e. during the first, second and/or third treatment period, the progestin is typically only administered in sub-periods having a duration of from ¼×28 to 1×28 days, preferably a duration of from ¼×28 to ¾×28 days, most preferably a duration of ½×28 days. Thus, the progestin may be administered for one or more sub-periods during the entire treatment period. As will be understood, the number of sub-periods (i.e. the number of times progestin treatment is commenced) within each treatment period will be highly dependent on the actual duration of the treatment period. Thus, if the treatment period in question is short, it may only be necessary to include a single sub-period of progestin treatment, whereas if the treatment period in question is relative long, it may be necessary to include two or more sub-periods of progestin treatment periods within the treatment period in question.

In general, the interval between initiation of sub-periods of progestin treatment (typically having a duration of from ¼×28 to 1×28 days, preferably a duration of from ¼×28 to ¾×28 days, most preferably a duration of ½×28 days), within each treatment period, should typically be in the order of 2×28 days to 4×28 days. For example, the interval between initiation of sub-periods of progestin treatment (typically having a duration of from ¼×28 to 1×28 days, preferably a duration of from ¼×28 to ¾×28 days, most preferably a duration of ½×28 days), within each treatment period, would typically be 2×28 days, 3×28 days or 4×28 days. Stated differently, during a given treatment period, a first sub-period of progestin treatment may be initiated 2×28 days, 2.5×28 days, 3×28 days, 3.5×28 days or 4×28 days after the treatment period in question is initiated. This first sub-period of progestin treatment may then be followed by a second sub-period of progestin treatment 2×28 days, 3×28 days or 4×28 days, preferably 3×28 days or 4×28 days after initiation of the first sub-period of progestin treatment. The above-mentioned intervals between sub-periods of progestin treatment may vary within each treatment period and/or may vary between the treatment periods. As will be understood, longer intervals between sub-periods of progestin treatment may be allowed during the second treatment period as compared to the first treatment period as the administered amount of estrogen is lower in the second treatment period as compared to the first treatment period.

In another aspect, the present invention relates to the use of a combination of an estrogen and a selective estrogen receptor modulator (SERM) compound having estrogen antagonistic activity in the endometrium for the manufacture of a medicament for the continuous treatment of diseases, conditions or symptoms associated with deficient endogenous levels of estrogen in a woman already receiving a therapeutically effective amount of an estrogen, wherein the administration pattern of said medicament comprises:
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) optionally continue treatment by administering to said woman, a therapeutically effective amount of an estrogen which is equal to or less than said third therapeutically effective amount of estrogen;
   wherein a selective estrogen receptor modulator (SERM) compound having estrogen antagonistic activity in the endometrium is administered during the entire treatment period or during a part of the treatment period.

In one embodiment, the selective estrogen receptor modulator (SERM) compound having estrogen antagonistic activity in the endometrium is administered during one or more sub-periods of the first treatment period according to the invention and/or during one or more sub-periods of said second treatment period according to the invention and/or during one or more sub-periods of said third treatment period according to the invention and/or during one or more sub-periods of the continuous treatment defined in step (iv).

The selective estrogen receptor modulator (SERM) compound having estrogen antagonistic activity in the endometrium can be selected from the group consisting of raloxifene and bazedoxifene.

In another embodiment, the invention relates to the use of a combination of an estrogen and a progestin or selective estrogen receptor modulator (SERM) compound having estrogen antagonistic activity in the endometrium for the manufacture of a medicament for the continuous treatment of diseases, conditions or symptoms associated with deficient endogenous levels of estrogen in a woman already receiving a therapeutically effective amount of an estrogen, wherein said woman is a post-menopausal woman or a non-hysterectomised woman.

In a further aspect, the invention relates to a method for the continuous treatment of diseases, conditions or symptoms associated with deficient endogenous levels of estrogen in a woman already receiving a therapeutically effective amount of an estrogen, said method comprising the steps of
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) optionally continue treatment by administering to said woman, a therapeutically effective amount of an estrogen which is equal to or less than said third therapeutically effective amount of estrogen.

As will be understood, all statements made above in connection with the aspect concerning use of an estrogen for the manufacture of a medicament for the continuous treatment of diseases, condition or symptoms associated with deficient endogenous levels of estrogen in a woman already receiving a therapeutically effective amount of an estrogen also apply to the aspect concerning the method for continuous treatment of the invention. Thus, all statements made above in connection with the duration of the various treatment periods, the amount of estrogen to be administered in the various treatment periods, ways of administering the estrogen, preferred estrogens to be administered, combination treatment with a progestin or a selective estrogen receptor modulator (SERM) compound having estrogen antagonistic activity in the endometrium, etc. apply mutatis mutandis to the aspect concerning the method for continuous treatment.

Pharmaceutical Compositions

As discussed above, the estrogen may be administered transdermally or via the oral route. When the estrogen, in particular estradiol hemihydrate, is administered via the oral route, the estrogen is preferably contained in an oral dosage unit, such as tablets (both swallowable-only and chewable forms), capsules, granules, granules enclosed in sachets, and pills. Hence, the oral dosage unit containing the estrogen, such as estradiol hemihydrate, may be in the form of a tablet, capsule, gelcap, granule, sachet or a pill. In a preferred embodiment of the invention, the oral dosage unit is in the form of a tablet or a capsule, in particular in the form of a tablet. Tablets may conveniently be coated with a suitable film-forming agent, e.g. hydroxypropylmethylcellulose.

The oral dosage unit containing the estrogen, in particular estradoil hemihydrate, may be formulated in any way conventional in the pharmaceutical art. In particular, the oral dosage unit may be formulated by a method comprising providing the estrogen, such as estradiol hemihydrate, in micronized form in said oral dosage unit, or sprayed from a solution onto particles of an inert carrier in admixture with one or more pharmaceutically acceptable excipients that promote dissolution of the estrogen, such as estradiol hemihydrate. Examples of suitable excipients include fillers, such as lactose, glucose or sucrose, sugar alcohols such as mannitol, starch such as corn or potato starch or modified starch; lubricants such as talc or magnesium stearate; and binders such as polyvinylpyrrolidone, cellulose derivatives, carboxymethyl cellulose, hydroxypropyl cellulose, hydroxypropylmethyl cellulose, methyl cellulose, or gelatin.

With respect to the estrogen, which may be a sparingly soluble substance, it is an advantage to provide it in micronized form or sprayed from a solution, e.g. in ethanol, onto the surface of inert carrier particles, such as described in EP 1 257 280. This has the added advantage of facilitating a more homogenous distribution of the estrogen throughout the composition. When the estrogen, such as estradiol hemihydrate, is provided in micronized form, it preferably has the following particle size distribution as determined under the microscope: 100% of the particles have a diameter of ≦15.0 μm, 99% of the particles have a diameter of ≦12.5 μm, 95% of the particles have a diameter of ≦10.0 μm, and 50% of the particles have a diameter of ≦3.0 μm. Independently of the particular formulation of the estrogen it is preferred, however, that the estrogen is formulated in such a way that at least 70% of the estrogen, such as estradiol, is dissolved within 30 minutes when the oral dosage unit is subjected to dissolution testing in 900 ml of water or 0.1N HCl at 37° C. using the USP XXIII Paddle Method II operated at a stirring rate of 50 rpm. Preferably, at least 80% of the estrogen, such as estradiol, is dissolved within 20 minutes when tested as described above. Such compositions are described in EP 1 257 280.

As discussed previously, a progestin, such as drospirenone, may be co-administered with the estrogen in one or more sub-periods during the various treatment periods. The progestin, such as drospirenone, may be formulated in a separate oral dosage unit or the progestin, such as drospirenone, may be formulated in the same oral dosage unit as the estrogen, such as estradiol hemihydrate. Either way, the progestin may be directly incorporated in the oral dosage units described above. However, it is preferred that the progestin, in particular drospirenone, is provided in micronised form or is sprayed from a solution onto particles of an inert carrier in admixture with one or more pharmaceutically acceptable excipients that promote dissolution of the progestin. Accordingly, if provided in micronised form the progestin, such as drospirenone, preferably fulfils the same particle size requirements as given above in connection with micronised estrogen. Independently of the particular formulation of the progestin it is preferred, however, that the progestin is formulated in such a way that at least 70% of the progestin, such as drospirenone, is dissolved within 30 minutes when the oral dosage unit is subjected to dissolution testing in 900 ml of water or 0.1N HCl at 37° C. using the USP XXIII Paddle Method II operated at a stirring rate of 50 rpm. Preferably, at least 80% of the progestin, such as drospirenone is dissolved within 20 minutes when tested as described above. Such compositions are described in EP 1 257 280.

Thus, the present invention also relates to pharmaceutical preparations comprising a number of separately packed and individually removable daily oral dosage units placed into a packaging unit. Such preparations can be adapted in such a way that ready-to-use packages for treatment during the individual treatment periods described herein are provided.

Thus, in a further aspect, the invention relates to a pharmaceutical preparation comprising a number of separately packed and individually removable daily oral dosage units placed into a packaging unit, wherein
(i) from 0.5×28 to 2×28 of said daily oral dosage units comprise from 0.9 to 1.1 mg estradiol; and
(ii) from 0.5×28 to 2×28 of said daily oral dosage units comprise from >0.4 to 0.65 mg estradiol; and
(iii) from 0.5×28 to 2×28 of said daily oral dosage units comprise from 0.2 to 0.4 mg estradiol.

In a specific embodiment, the daily oral dosage units specified in (i) of the pharmaceutical preparation according to the invention comprise from 0.9 to 1.05 mg estradiol, or from 0.9 to 1.0 mg estradiol, or from 0.95 to 1.1 mg estradiol, or from 0.95 to 1.05 mg estradiol, or about 0.9 mg estradiol, or about 0.95 mg, or about 1 mg, or about 1.05 mg, or about 1.1 mg estradiol, preferably about 1 mg estradiol. In a further specific embodiment, the number of daily oral dosage units of the pharmaceutical preparation according to the invention specified in (i) above is 0.5×28 to 1.5×28, or 0.5×28 to 1×28, or about 0.5×28, or about 1×28, or about 1.5×28, or about 2×28, preferably 1×28.

In another specific embodiment, the daily oral dosage units specified in (ii) of the pharmaceutical preparation according to the invention comprise from >0.4 to 0.6 mg estradiol, or from 0.45 to 0.6 mg estradiol, or from 0.5 to 0.6 mg estradiol, or from 0.5 to 0.55 mg estradiol, or about 0.45 mg estradiol, or about 0.5 mg, or about 0.55 mg, or about 0.6 mg, or about 0.65 mg estradiol, preferably about 0.5 mg estradiol. In a further specific embodiment, the number of daily oral dosage units of the pharmaceutical preparation according to the invention specified in (ii) above is 0.5×28 to 1.5×28, or 0.5×28 to 1×28, or about 0.5×28, or about 1×28, or about 1.5×28, or about 2×28, preferably 1×28.

In a further specific embodiment, the daily oral dosage units specified in (iii) of the pharmaceutical preparation according to the invention comprise from 0.2 to 0.35 mg estradiol, or from 0.25 to 0.4 mg estradiol, or from 0.25 to 0.35 mg estradiol, or about 0.2 mg estradiol, or about 0.25 mg, or about 0.3 mg, or about 0.35 mg, or about 0.4 mg estradiol, preferably about 0.3 mg estradiol. In yet a further embodiment, the number of daily oral dosage units specified in (iii) of the pharmaceutical preparation is 0.5×28 to 1.5×28, or for 1×28 to 2×28, preferably for 1×28 days.

In a second embodiment, the invention relates to a pharmaceutical preparation comprising a number of separately packed and individually removable daily oral dosage units placed into a packaging unit, wherein
(ii) from 1×28 to 2×28 of said daily oral dosage units comprise from >0.4 to 0.65 mg estradiol; and
(iii) from 1×28 to 2×28 of said daily oral dosage units comprise from 0.2 to 0.4 mg estradiol.

The daily oral dosage units specified in (ii) of the pharmaceutical preparation according to the invention can comprise about 0.5 mg estradiol. The number of daily oral dosage units specified in (ii) of the pharmaceutical preparation according to the invention can be 1.5×28. The daily oral dosage units of the pharmaceutical preparation specified in (iii) above can comprise about 0.3 mg estradiol. The number of daily oral dosage units specified in (iii) above can be 1.5×28.

In a third embodiment, the invention relates to a pharmaceutical preparation comprising a number of separately packed and individually removable daily oral dosage units placed into a packaging unit, wherein
(i) from 1×28 to 2×28 of said daily oral dosage units comprise from 0.9 to 1.1 mg estradiol; and
(iii) from 1×28 to 2×28 of said daily oral dosage units comprise from 0.2 to 0.4 mg estradiol.

The daily oral dosage units specified in (i) above can comprise about 1 mg estradiol. The number of daily oral dosage units specified in (i) above can be 1.5×28. The daily oral dosage units of the pharmaceutical preparation specified in (iii) above can comprise about 0.3 mg estradiol. The number of daily oral dosage units specified in (iii) above can be 1.5×28.

The pharmaceutical preparation according to the invention can further comprise a progestin or a selective estrogen receptor modulator (SERM) compound having estrogen antagonistic activity in the endometrium. The progestins and the SERMs are defined above. In a specific embodiment, one, some or all of the daily oral dosage units of the pharmaceutical preparation according to the invention specified in (i), i.e. daily oral dosage units comprising from 0.9 to 1.1 mg estradiol, further comprises a progestin or a selective estrogen receptor modulator (SERM) compound having estrogen antagonistic activity in the endometrium. In a further embodiment, one, some or all of the daily oral dosage units of the pharmaceutical preparation according to the invention specified in (ii), i.e. daily oral dosage units comprising from >0.4 to 0.65 mg estradiol, further comprises a progestin or a selective estrogen receptor modulator (SERM) compound having estrogen antagonistic activity in the endometrium. In yet a further embodiment, one, some or all of the daily oral dosage units of the pharmaceutical preparation according to the invention specified in (iii), i.e. daily oral dosage units comprising 0.2 to 0.4 mg estradiol, further comprises a progestin or a selective estrogen receptor modulator (SERM) compound having estrogen antagonistic activity in the endometrium.

In one embodiment, the invention relates to a pharmaceutical preparation comprising a number of separately packed transdermal patches placed into a packaging unit, wherein
(i) one or more of said patches delivers a dose of from 35 to 55 μg estradiol per day; and
(ii) one or more of said patches delivers a dose of from 20 to 30 μg estradiol per day; and
(iii) one or more of said patches delivers a dose of from 12 to 16 μg estradiol per day.

In another embodiment, the one or more patches specified in (i) above delivers a dose of about 50 μg estradiol per day or about 37.5 μg estradiol per day. In a further embodiment, the one or more patches specified in (ii) above delivers a dose of about 25 μg estradiol per day. In yet a further embodiment, the one or more patches specified in (iii) above delivers a dose of about 14 μg estradiol per day. In still a further embodiment, the number of patches specified in (i), (ii) and (iii) above are independently in the ranges of from 2-8, such as 2, 3, 4, 5, 6, 7 or 8, preferably 3, 4 or 5, more preferably 4.

In yet another embodiment, the invention relates to a pharmaceutical preparation comprising a number of separately packed transdermal patches placed into a packaging unit, wherein
(ii) one or more of said patches delivers a dose of from 20 to 30 μg estradiol per day; and
(iii) one or more of said patches delivers a dose of from 12 to 16 μg estradiol per day.

In a specific embodiment, the one or more patches specified in (ii) above delivers a dose of about 25 μg estradiol per day. In another specific embodiment, the one or more patches specified in (iii) above delivers a dose of about 14 μg estradiol per day. In a further embodiment, the number of patches specified in (ii) and (iii) above are independently in the ranges of from 4-8, such as 4, 5, 6, 7 or 8, preferably 5, 6 or 7, more preferably 6.

In still another embodiment, the invention relates to a pharmaceutical preparation comprising a number of separately packed transdermal patches placed into a packaging unit, wherein
(i) one or more of said patches delivers a dose of from 35 to 55 μg estradiol per day; and
(iii) one or more of said patches delivers a dose of from 12 to 16 μg estradiol per day.

In one embodiment, the one or more patches specified in (i) above delivers a dose of about 50 μg estradiol per day or about 37.5 μg estradiol per day. In another embodiment, the one or more patches specified in (iii) above delivers a dose of about 14 μg estradiol per day. In yet another embodiment, the number of patches specified in (ii) and (iii) above are independently in the ranges of from 4-8, such as 4, 5, 6, 7 or 8, preferably 5, 6 or 7, more preferably 6.

As outlined above the pharmaceutical preparation according to the invention can comprise a number of separately packed transdermal patches. In one embodiment of the invention one, some or all of the transdermal patches specified in (i), i.e. patches delivering a dose of from 35 to 55 μg estradiol per day, further comprises a progestin or a selective estrogen receptor modulator (SERM) compound having estrogen antagonistic activity in the endometrium. In another embodiment of the invention one, some or all of the transdermal patches specified in (ii), i.e. patches delivering a dose of from 20 to 30 μg estradiol per day, further comprises a progestin or a selective estrogen receptor modulator (SERM) compound having estrogen antagonistic activity in the endometrium. In still another embodiment of the invention, one, some or all of the transdermal patches specified in (iii), i.e patches delivering a dose of from 12 to 16 μg estradiol per day, further comprises a progestin or a selective estrogen receptor modulator (SERM) compound having estrogen antagonistic activity in the endometrium.

In a similar way as described above, the estrogen in the pharmaceutical preparation according to the invention is preferably estradiol or a salt, hydrate or a therapeutically acceptable derivative thereof, in particular estradiol hemihydrate. Likewise, the progestin may be selected from the group consisting of levo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desorgestrel, norgestimate, gestodene, tibolone, cyproterone acetate and drospirenone. As discussed previously, the progestin is preferably drospirenone.

A packaging unit comprising the daily dosage units described above may be prepared in a manner analogous to that of making oral contraceptives or hormone replacement regimens. This may for instance be a conventional blister pack or any other form known for this purpose, for instance a pack comprising the appropriate number of dosage units (in this case at least 28, or for particular applications, a multiple of 28) in a sealed blister pack with a cardboard, paperboard, foil or plastic backing and enclosed in a suitable cover. Each blister container may conveniently be numbered or otherwise marked.

When transdermal formulations are considered, they may be prepared in the form of matrices or membranes or as fluid or viscous formulations in oil or hydrogels. For transdermal patches, an adhesive which is compatible with the skin should be included, such as polyacrylate, a silicone adhesive or polyisobutylene, as well as a foil made of, e.g. polyethylene, polypropylene, ethylene vinylacetate, polyvinylchloride, polyvinylidene chloride or polyester, and a removable protective foil made from, e.g., polyester or paper coated with silicone or a fluoropolymer. For the preparation of transdermal solutions or gels, water or organic solvents or mixtures thereof may be used. Transdermal gels may furthermore contain one or more suitable gelling agents or thickeners such as silicone, tragacanth, starch or starch derivatives, cellulose or cellulose derivatives or polyacrylic acids or derivatives thereof. Transdermal formulations may also suitably contain one or more substances that enhance absorption though the skin, such as bile salts or derivatives thereof and/or phospholipids. Suitable transdermal formulations may, for instance, be made in a manner analogous to that described in WO 94/04157 for 3-ketodesogestrel. Alternatively, transdermal formulations may be prepared according to a method disclosed in, e.g., B W Barry, “Dermatological Formulations, Percutaneous Absorption”, Marcel Dekker Inc., New York-Basel, 1983, or Y W Chien, “Transdermal Controlled Systemic Medications”, Marcel Dekker Inc., New York-Basel, 1987.

As will understood by the skilled person, transdermal formulations such as estrogen-containing patches will be worn for a certain period of time, e.g. 3, 4, 5, 6, 7 or up till 14 days (which is typically considerably shorter than the first treatment period), after which the patch needs to be replaced with a new one.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

Transdermal Delivery of Estradiol

### Example 1

A packaging unit comprising
(i) 4 transdermal patches capable of delivering 37.5 μg/day for 1 week each;
(ii) 4 transdermal patches capable of delivering 25 μg/day for 1 week each;
(iii) 4 transdermal patches capable of delivering 14 μg/day for 1 week each.

### Example 2

A packaging unit comprising
(i) 2 transdermal patches capable of delivering 37.5 μg/day for 2 weeks each;
(ii) 2 transdermal patches capable of delivering 25 μg/day for 2 weeks each;
(iii) 2 transdermal patches capable of delivering 14 μg/day for 2 weeks each.

### Example 3

A packaging unit comprising
(i) 4 transdermal patches capable of delivering 50 μg/day for 1 week each;
(ii) 4 transdermal patches capable of delivering 25 μg/day for 1 week each;
(iii) 4 transdermal patches capable of delivering 14 μg/day for 1 week each.

### Example 4

A packaging unit comprising
(i) 2 transdermal patches capable of delivering 50 μg/day for 2 weeks each;
(ii) 2 transdermal patches capable of delivering 25 μg/day for 2 weeks each;
(iii) 2 transdermal patches capable of delivering 14 μg/day for 2 weeks each.

### Example 5

A packaging unit comprising
(i) 6 transdermal patches capable of delivering 25 μg/day for 1 week each;
(ii) 6 transdermal patches capable of delivering 14 μg/day for 1 week each.

### Example 6

A packaging unit comprising
(i) 3 transdermal patches capable of delivering 25 μg/day for 2 weeks each;
(ii) 3 transdermal patches capable of delivering 14 μg/day for 2 weeks each.

### Example 7

A packaging unit comprising
(i) 6 transdermal patches capable of delivering 37 μg/day for 1 week each;
(ii) 6 transdermal patches capable of delivering 14 μg/day for 1 week each.

### Example 8

A packaging unit comprising
(i) 3 transdermal patches capable of delivering 37 μg/day for 2 weeks each;
(ii) 3 transdermal patches capable of delivering 14 μg/day for 2 weeks each.

### Example 9

A packaging unit comprising
(i) 6 transdermal patches capable of delivering 50 μg/day for 1 week each;
(ii) 6 transdermal patches capable of delivering 14 μg/day for 1 week each.

### Example 10

A packaging unit comprising
(i) 3 transdermal patches capable of delivering 50 μg/day for 2 weeks each;
(ii) 3 transdermal patches capable of delivering 14 μg/day for 2 weeks each.

Oral Delivery of Estradiol

### Example 11

One or more blister packs containing a total of:
(i) 28 individually removable daily oral dosage units containing 1 mg estradiol as estradiol hemihydrate;
(ii) 28 individually removable daily oral dosage units containing 0.5 mg estradiol as estradiol hemihydrate; and
(iii) 28 individually removable daily oral dosage units containing 0.3 mg estradiol as estradiol hemihydrate.

The one or more blister packs are placed into a packaging unit.

### Example 12

One or more blister packs containing a total of:
(i) 42 individually removable daily oral dosage units containing 0.5 mg estradiol as estradiol hemihydrate; and
(ii) 42 individually removable daily oral dosage units containing 0.3 mg estradiol as estradiol hemihydrate.

The one or more blister packs are placed into a packaging unit.

### Example 13

One or more blister packs containing a total of:
(i) 42 individually removable daily oral dosage units containing 1 mg estradiol as estradiol hemihydrate; and
(ii) 42 individually removable daily oral dosage units containing 0.3 mg estradiol as estradiol hemihydrate.

The one or more blister packs are placed into a packaging unit.

### Example 14

One or more blister packs containing a total of:
(i) 28 individually removable daily oral dosage units containing 0.625 mg CEE;
(ii) 28 individually removable daily oral dosage units containing 0.3 mg CEE; and
(iii) 28 individually removable daily oral dosage units containing 0.15 mg CEE.

The one or more blister packs are placed into a packaging unit.

### Example 15

One or more blister packs containing a total of:
(i) 42 individually removable daily oral dosage units containing 0.3 mg CEE; and
(ii) 42 individually removable daily oral dosage units containing 0.15 mg CEE.

The one or more blister packs are placed into a packaging unit.

### Example 16

One or more blister packs containing a total of:
(i) 42 individually removable daily oral dosage units containing 0.625 mg CEE; and
(ii) 42 individually removable daily oral dosage units containing 0.15 mg CEE.

The one or more blister packs are placed into a packaging unit.

1. A method of an estrogen for the manufacture of a medicament for the continuous treatment of diseases, conditions or symptoms associated with deficient endogenous levels of estrogen in a woman already receiving a therapeutically effective amount of an estrogen, performed wherein the administration pattern of said medicament comprises:
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) optionally continue treatment by administering to said woman, a therapeutically effective amount of an estrogen which is equal to or less than said third therapeutically effective amount of estrogen.

2. A method according to claim 1, performed wherein said first therapeutically effective amount of estrogen is administered orally.

3. A method according to claim 2, performed wherein said first therapeutically effective amount of estrogen is administered once daily during the first treatment period.

4. A method according to claim 1, performed wherein said estrogen is estradiol or a salt, hydrate or a therapeutically acceptable derivative thereof.

5. A method according to claim 4, performed wherein said estrogen is estradiol hemihydrate.

6. A method according to claim 1, performed wherein said first therapeutically effective amount of estrogen corresponds to a therapeutically equivalent amount of estradiol in the range of from >0.75 to 1.5 mg per day, preferably in the range of from >0.75 to 1.25 mg per day, more preferably in the range of from 0.9 to 1.1 mg per day, most preferably about 1 mg per day.

7. A method according to claim 1, performed wherein said first therapeutically effective amount of estrogen is administered transdermally.

8. A method according to claim 7, performed wherein said first therapeutically effective amount of estrogen corresponds to a therapeutically equivalent amount of estradiol in the range of from >32.5 to 75 μg per day, preferably in the range of from >32.5 to 62.5 μg per day, more preferably in the range of from 35 to 55 μg per day, most preferably about 37.5 μg per day or about 50 μg per day.

9. A method according to claim 1, performed wherein said first treatment period is continued for 0.5×28 to 6×28 days, preferably for 0.5×28 to 4×28 days, more preferably for 0.5×28 to 2×28 days.

10. A method according to claim 9, performed wherein said first treatment period is continued for 0.5×28 days, 1×28 days, 1.5×28 days or 2×28 days, most preferably for 1×28 days or 1.5×28 days.

11. A method according to claim 1, performed wherein step (i) is omitted.

12. A method according to claim 1, performed wherein said second therapeutically effective amount of estrogen is administered orally.

13. A method according to claim 12, performed wherein said second therapeutically effective amount of estrogen is administered once daily during the second treatment period.

14. A method according to claim 12, performed wherein said estrogen is estradiol or a salt, hydrate or a therapeutically acceptable derivative thereof.

15. A method according to claim 14, performed wherein said estrogen is estradiol hemihydrate.

16. A method according to claim 12, performed wherein said second therapeutically effective amount of estrogen corresponds to a therapeutically equivalent amount of estradiol in the range of from >0.4 to 0.75 mg per day, preferably in the range of from >0.4 to 0.65 mg per day, more preferably in the range of from >0.4 to 0.55 mg per day, even more preferably in the range of from 0.45 to 0.55 mg per day, most preferably about 0.5 mg per day.

17. A method according to claim 1, performed wherein said second therapeutically effective amount of estrogen is administered transdermally.

18. A method according to claim 17, performed wherein said second therapeutically effective amount of estrogen corresponds to a therapeutically equivalent amount of estradiol of from >20 to 32.5 μg per day, preferably in the range of from >20 to 30 μg per day, more preferably in the range of from 22.5 to 27.5 μg per day, most preferably about 25 μg per day.

19. A method according to claim 1, performed wherein said second treatment period is continued for 0.5×28 to 6×28 days, preferably for 0.5×28 to 4×28 days, more preferably for 0.5×28 to 2×28 days.

20. A method according to claim 19, performed wherein said second treatment period is continued for 0.5×28 days, 1×28 days, 1.5×28 days or 2×28 days, most preferably for 1×28 days or 1.5×28 days.

21. A method according to claim 1, performed wherein step (ii) is omitted.

22. A method according to claim 1, performed wherein said third therapeutically effective amount of estrogen is administered orally.

23. A method according to claim 22, performed wherein said third therapeutically effective amount of estrogen is administered once daily during the third treatment period.

24. A method according to claim 22, performed wherein said estrogen is estradiol or a salt, hydrate or a therapeutically acceptable derivative thereof.

25. A method according to claim 24, performed wherein said estrogen is estradiol hemihydrate.

26. A method according to claim 22, performed wherein said third therapeutically effective amount of estrogen corresponds to a therapeutically equivalent amount of estradiol in the range of from 0.05 to 0.4 mg per day, preferably in the range of from 0.1 to 0.4 mg per day, more preferably in the range of from 0.2 to 0.4 mg per day, even more preferably in the range of from 0.25 to 0.35 mg per day, most preferably about 0.3 mg per day.

27. A method according to claim 1, performed wherein said third therapeutically effective amount of estrogen is administered transdermally.

28. A method according to claim 27, performed wherein said third therapeutically effective amount of estrogen corresponds to a therapeutically equivalent amount of estradiol in the range of from 2.5 to 20 μg per day, preferably in the range of from 5 to 20 μg per day, more preferably in the range of from 10 to 20 μg per day, even more preferably in the range of from 12 to 16 μg per day, most preferably about 14 μg per day.

29. A method according to claim 1, performed wherein said third treatment period is continued for 0.5×28 to 6×28 days, preferably for 0.5×28 to 4×28 days, more preferably for 0.5×28 to 2×28 days.

30. A method according to claim 29, performed wherein said third treatment period is continued for 0.5×28 days, 1×28 days, 1.5×28 days or 2×28 days, most preferably for 1×28 days or 1.5×28 days.

31. A method of estradiol for the manufacture of a medicament for the continuous treatment of diseases, conditions or symptoms associated with deficient endogenous levels of estrogen in a woman already receiving a therapeutically effective amount of an estrogen, performed wherein the administration pattern of said medicament comprises:
(i) administering to said woman a daily oral dose of from 0.9 to 1.1 mg estradiol for 0.5×28 to 2×28 days; and then
(ii) administering to said woman a daily oral dose of from >0.4 to 0.65 mg estradiol for 0.5×28 to 2×28 days; and then;
(iii) administering to said woman a daily oral dose of from 0.2 to 0.4 mg estradiol for 0.5×28 to 2×28 days; and
(iv) optionally continue treatment by administering to said woman, a therapeutically effective amount of estradiol which is equal to or less than the dose specified in (iii).

32. A method according to claim 31, performed wherein said daily oral dose specified in (i) is about 1 mg estradiol.

33. A method according to claim 31, performed wherein said daily oral dose specified in (i) is administered for 0.5×28 to 1.5×28 days, preferably for 1×28 days.

34. A method according to claim 31, performed wherein said daily oral dose specified in (ii) is about 0.5 mg estradiol.

35. A method according to claim 31, performed wherein said daily oral dose specified in (ii) is administered for 0.5×28 to 1.5×28 days, preferably for 1×28 days.

36. A method according to claim 31, performed wherein said daily oral dose specified in (iii) is about 0.3 mg estradiol.

37. A method according to claim 31, performed, wherein said daily oral dose specified in (iii) is administered for 0.5×28 to 1.5×28 days, preferably for 1×28 days.

38. A method of estradiol for the manufacture of a medicament for the continuous treatment of diseases, conditions or symptoms associated with deficient endogenous levels of estrogen in a woman already receiving a therapeutically effective amount of an estrogen, performed wherein the administration pattern of said medicament comprises:
(ii) administering to said woman a daily oral dose of from >0.4 to 0.65 mg estradiol for 1×28 to 2×28 days; and then;
(iii) administering to said woman a daily oral dose of from 0.2 to 0.4 mg estradiol for 1×28 to 2×28 days; and
(iv) optionally continue treatment by administering to said woman, a therapeutically effective amount of estradiol which is equal to or less than the dose specified in (iii).

39. A method according to claim 38, performed wherein said daily oral dose specified in (ii) is about 0.5 mg estradiol.

40. A method according to claim 38, performed wherein said daily oral dose specified in (ii) is administered for 1.5×28 days.

41. A method according to any of claims 38, performed wherein said daily oral dose specified in (iii) is about 0.3 mg estradiol.

42. A method according to any of claims 38, performed wherein said daily oral dose specified in (iii) is administered for 1.5×28 days.

43. A method of estradiol for the manufacture of a medicament for the continuous treatment of diseases, conditions or symptoms associated with deficient endogenous levels of estrogen in a woman already receiving a therapeutically effective amount of an estrogen, performed wherein the administration pattern of said medicament comprises:
(i) administering to said woman a daily oral dose of from 0.9 to 1.1 mg estradiol for 1×28 to 2×28 days; and then;
(iii) administering to said woman a daily oral dose of from 0.2 to 0.4 mg estradiol for 1×28 to 2×28 days; and
(iv) optionally continue treatment by administering to said woman, a therapeutically effective amount of estradiol which is equal to or less than the dose specified in (iii).

44. A method according to claim 43, performed wherein said daily oral dose specified in (i) is about 1 mg estradiol.

45. A method according to claim 43, performed wherein said daily oral dose specified in (i) is administered for 1.5×28 days.

46. A method according to claim 43, performed wherein said daily oral dose specified in (iii) is about 0.3 mg estradiol.

47. A method according to claim 43, performed wherein said daily oral dose specified in (iii) is administered for 1.5×28 days.

48. A method of estradiol for the manufacture of a medicament for the continuous treatment of diseases, conditions or symptoms associated with deficient endogenous levels of estrogen in a woman already receiving a therapeutically effective amount of an estrogen, performed wherein the administration pattern of said medicament comprises:
(i) administering to said woman a transdermal dose of from 35 to 55 μg estradiol per day for 0.5×28 to 2×28 days; and then
(ii) administering to said woman a transdermal dose of from 20 to 30 μg estradiol per day for 0.5×28 to 2×28 days; and then;
(iii) administering to said woman a transdermal dose of from 12 to 16 μg estradiol per day for 0.5×28 to 2×28 days; and
(iv) optionally continue treatment by administering to said woman, a therapeutically effective amount of estradiol which is equal to or less than the dose specified in (iii).

49. A method according to claim 48, performed wherein said daily dose specified in (i) is about 50 μg estradiol per day.

50. A method according to claim 48, performed wherein said daily dose specified in (i) is about 37.5 μg estradiol per day.

51. A method according to claim 48, performed wherein said daily dose specified in (i) is administered for 0.5×28 to 1.5×28 days, preferably for 1×28 days.

52. A method according to claim 48, performed wherein said daily dose specified in (ii) is about 25 μg estradiol per day.

53. A method according to claim 48, performed wherein said daily dose specified in (ii) is administered for 0.5×28 to 1.5×28 days, preferably for 1×28 days.

54. A method according to claim 48, performed wherein said daily dose specified in (iii) is about 14 μg estradiol per day.

55. A method according to claim 48, performed wherein said daily dose specified in (iii) is administered for 0.5×28 to 1.5×28 days, preferably for 1×28 days.

56. A method of estradiol for the manufacture of a medicament for the continuous treatment of diseases, conditions or symptoms associated with deficient endogenous levels of estrogen in a woman already receiving a therapeutically effective amount of an estrogen, performed wherein the administration pattern of said medicament comprises:
(ii) administering to said woman a transdermal dose of from 20 to 30 μg estradiol per day for 1×28 to 2×28 days; and then;
(iii) administering to said woman a transdermal dose of from 12 to 16 μg estradiol per day for 1×28 to 2×28 days; and
(iv) optionally continue treatment by administering to said woman, a therapeutically effective amount of estradiol which is equal to or less than the dose specified in (iii).

57. A method according to claim 56, performed wherein said daily dose specified in (ii) is about 25 μg estradiol per day.

58. A method according to claim 56, performed wherein said daily dose specified in (ii) is administered for 1.5×28 days.

59. A method according to claim 56, performed wherein said daily dose specified in (iii) is about 14 μg estradiol per day.

60. A method use according to claim 56, performed wherein said daily dose specified in (iii) is administered for 1.5×28 days.

61. A method of estradiol for the manufacture of a medicament for the continuous treatment of diseases, conditions or symptoms associated with deficient endogenous levels of estrogen in a woman already receiving a therapeutically effective amount of an estrogen, performed wherein the administration pattern of said medicament comprises:
(i) administering to said woman a transdermal dose of from 35 to 55 μg estradiol per day for 1×28 to 2×28 days; and then
(iii) administering to said woman a transdermal dose of from 12 to 16 μg estradiol per day for 1×28 to 2×28 days; and
(iv) optionally continue treatment by administering to said woman, a therapeutically effective amount of estradiol which is equal to or less than the dose specified in (iii).

62. A method according to claim 61, performed wherein the daily dose specified in (i) is about 50 μg estradiol per day.

63. A method according to claim 61, performed wherein the daily dose specified in (i) is about 37.5 μg estradiol per day.

64. A method according to any of claims 61, performed wherein said daily dose specified in (i) is administered for 1.5×28 days.

65. A method according to claim 61, performed wherein said daily dose specified in (iii) is about 14 μg estradiol per day.

66. A method according to claim 61, performed wherein said daily dose specified in (iii) is administered for 1×28 days.

67. A method according to claim 1, performed wherein said woman is a post-menopausal woman.

68. A method according to claim 1, performed wherein said woman is a hysterectomised woman.

69. A method according to claim 1, performed wherein a progestin is administered during the entire treatment period or during a part of the treatment period.

70. A method according to claim 69, performed wherein said progestin is selected from the group consisting of levo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desorgestrel, norgestimate, gestodene, tibolone, cyproterone acetate and drospirenone.

71. A method according to claim 70, performed wherein said progestin is drospirenone.

72. A method according to claim 1, performed wherein a selective estrogen receptor modulator (SERM) compound having estrogen antagonistic activity in the endometrium is administered during the entire treatment period or during a part of the treatment period.

73. A method according to claim 72, performed wherein the selective estrogen receptor modulator (SERM) compound is selected from the group consisting of raloxifene and bazedoxifene.

74. A method according to claim 1, performed wherein said woman is a post-menopausal woman.

75. A method according to-, claim 69, performed wherein said woman is a non-hysterectomised woman.

76. A method for the continuous treatment of diseases, conditions or symptoms associated with deficient endogenous levels of estrogen in a woman already receiving a therapeutically effective amount of an estrogen, said method comprising the steps of
(i) administering to said woman a first therapeutically effective amount of an estrogen during a first treatment period, wherein said first therapeutically effective amount of an estrogen is equal to or less than the therapeutically effective amount of an estrogen already administered to said woman; and
(ii) after completion of the first treatment period, administering to said woman a second therapeutically effective amount of an estrogen during a second treatment period, where said second therapeutically effective amount of estrogen is less than said first therapeutically effective amount of estrogen; and
(iii) after completion of the second treatment period, administering to said woman a third therapeutically effective amount of an estrogen during a third treatment period, where said third therapeutically effective amount of estrogen is less than said second therapeutically effective amount of estrogen; and
(iv) optionally continue treatment by administering to said woman, a therapeutically effective amount of an estrogen which is equal to or less than said third therapeutically effective amount of estrogen.

77. The method according to claim 76, wherein said first therapeutically effective amount of estrogen is administered orally.

78. A pharmaceutical preparation comprising a number of separately packed and individually removable daily oral dosage units placed into a packaging unit, wherein
(i) from 0.5×28 to 2×28 of said daily oral dosage units comprise from 0.9 to 1.1 mg estradiol; and
(ii) from 0.5×28 to 2×28 of said daily oral dosage units comprise from >0.4 to 0.65 mg estradiol; and
(iii) from 0.5×28 to 2×28 of said daily oral dosage units comprise from 0.2 to 0.4 mg estradiol.

79. The preparation according to claim 78, wherein said daily oral dosage units specified in (i) comprise about 1 mg estradiol.

80. The preparation according to claim 78, wherein the number of daily oral dosage units specified in (i) is 0.5×28 to 1.5×28, preferably 1×28.

81. The preparation according to claim 78, wherein said daily oral dosage units specified in (ii) comprise about 0.5 mg estradiol.

82. The preparation according to claim 78, wherein the number of daily oral dosage units specified in (ii) is 0.5×28 to 1.5×28, preferably 1×28.

83. The preparation according to claim 78, wherein said daily oral dosage units specified in (iii) comprise about 0.3 mg estradiol.

84. The preparation according to claim 78, wherein the number of daily oral dosage units specified in (iii) is 0.5×28 to 1.5×28, preferably 1×28.

85. A pharmaceutical preparation comprising a number of separately packed and individually removable daily oral dosage units placed into a packaging unit, wherein
(ii) from 1×28 to 2×28 of said daily oral dosage units comprise from >0.4 to 0.65 mg estradiol; and
(iii) from 1×28 to 2×28 of said daily oral dosage units comprise from 0.2 to 0.4 mg estradiol.

86. The preparation according to claim 85, wherein said daily oral dosage units specified in (ii) comprise about 0.5 mg estradiol.

87. The preparation according to claim 85, wherein the number of daily oral dosage units specified in (ii) is 1.5×28.

88. The preparation according to claim 85, wherein said daily oral dosage units specified in (iii) comprise about 0.3 mg estradiol.

89. The preparation according to claim 85, wherein the number of daily oral dosage units specified in (iii) is 1.5×28.

90. A pharmaceutical preparation comprising a number of separately packed and individually removable daily oral dosage units placed into a packaging unit, wherein
(i) from 1×28 to 2×28 of said daily oral dosage units comprise from 0.9 to 1.1 mg estradiol; and
(iii) from 1×28 to 2×28 of said daily oral dosage units comprise from 0.2 to 0.4 mg estradiol.

91. The preparation according to claim 90, wherein said daily oral dosage units specified in (i) comprise about 1 mg estradiol.

92. The preparation according to claim 90, wherein the number of daily oral dosage units specified in (i) is 1.5×28.

93. The preparation according to claim 90, wherein said daily oral dosage units specified in (iiii) comprise about 0.3 mg estradiol.

94. The preparation according to claim 90, wherein the number of daily oral dosage units specified in (iii) is 1.5×28.

95. The preparation according to claim 78, wherein one, some or all of the daily oral dosage units specified in (i) further comprises a progestin.

96. The preparation according to claim 78, wherein one, some or all of the daily oral dosage units specified in (ii) further comprises a progestin.

97. The preparation according to claim 78, wherein one, some or all of the daily oral dosage units specified in (iii) further comprises a progestin.

98. The preparation according to claim 78, wherein one, some or all of the daily oral dosage units specified in (i) further comprises a selective estrogen receptor modulator (SERM) compound having estrogen antagonistic activity in the endometrium.

99. The preparation according to claim 78, wherein one, some or all of the daily oral dosage units specified in (ii) further comprises a selective estrogen receptor modulator (SERM) compound having estrogen antagonistic activity in the endometrium.

100. The preparation according to claim 78, wherein one, some or all of the daily oral dosage units specified in (iii) further comprises a selective estrogen receptor modulator (SERM) compound having estrogen antagonistic activity in the endometrium.

101. The preparation according to claim 78, wherein the selective estrogen receptor modulator (SERM) compound is selected from the group consisting of raloxifene and bazedoxifene.

102. A pharmaceutical preparation comprising a number of separately packed transdermal patches placed into a packaging unit, wherein
(i) one or more of said patches delivers a dose of from 35 to 55 μg estradiol per day; and
(ii) one or more of said patches delivers a dose of from 20 to 30 μg estradiol per day; and
(iii) one or more of said patches delivers a dose of from 12 to 16 μg estradiol per day.

103. The preparation according to claim 102, wherein the one or more patches specified in (i) delivers a dose of about 50 μg estradiol per day.

104. The preparation according to claim 102, wherein the one or more patches specified in (i) delivers a dose of about 37.5 μg estradiol per day.

105. The preparation according to claim 102, wherein the one or more patches specified in (ii) delivers a dose of about 25 μg estradiol per day.

106. The preparation according to claim 102, wherein the one or more patches specified in (iii) delivers a dose of about 14 μg estradiol per day.

107. The preparation according to claim 102, wherein the number of patches specified in (i), (ii) and (iii) are independently in the ranges of from 2-8, such as 2, 3, 4, 5, 6, 7 or 8, preferably 3, 4 or 5, more preferably 4.

108. A pharmaceutical preparation comprising a number of separately packed transdermal patches placed into a packaging unit, wherein
(ii) one or more of said patches delivers a dose of from 20 to 30 μg estradiol per day; and
(iii) one or more of said patches delivers a dose of from 12 to 16 μg estradiol per day.

109. The preparation according to claim 108, wherein the one or more patches specified in (ii) delivers a dose of about 25 μg estradiol per day.

110. The preparation according to claim 108, wherein the one or more patches specified in (iii) delivers a dose of about 14 μg estradiol per day.

111. The preparation according to claim 108, wherein the number of patches specified in (ii) and (iii) are independently in the ranges of from 4-8, such as 4, 5, 6, 7 or 8, preferably 5, 6 or 7, more preferably 6.

112. A pharmaceutical preparation comprising a number of separately packed transdermal patches placed into a packaging unit, wherein
(i) one or more of said patches delivers a dose of from 35 to 55 μg estradiol per day; and
(iii) one or more of said patches delivers a dose of from 12 to 16 μg estradiol per day.

113. The preparation according to claim 112, wherein the one or more patches specified in (i) delivers a dose of about 50 μg estradiol per day.

114. The preparation according to claim 112, wherein the one or more patches specified in (i) delivers a dose of about 37.5 μg estradiol per day.

115. The preparation according to claim 112, wherein the one or more patches specified in (iii) delivers a dose of about 14 μg estradiol per day.

116. The preparation according to claim 112, wherein the number of patches specified in (ii) and (iii) are independently in the ranges of from 4-8, such as 4, 5, 6, 7 or 8, preferably 5, 6 or 7, more preferably 6.

117. The preparation according to claim 102, wherein one, some or all of the patches specified in (i) further comprises a progestin.

118. The preparation according to claim 102, wherein one, some or all of the patches specified in (ii) further comprises a progestin.

119. The preparation according to claim 102, wherein one, some or all of the patches specified in (iii) further comprises a progestin.

120. The preparation according to claim 102, wherein one, some or all of the patches specified in (i) further comprises a selective estrogen receptor modulator (SERM) compound having estrogen antagonistic activity in the endometrium.

121. The preparation according to claim 102, wherein one, some or all of the patches specified in (ii) further comprises a selective estrogen receptor modulator (SERM) compound having estrogen antagonistic activity in the endometrium.

122. The preparation according to claim 102, wherein one, some or all of the patches specified in (iii) further comprises a selective estrogen receptor modulator (SERM) compound having estrogen antagonistic activity in the endometrium.

123. The preparation according to, claim 120, wherein the selective estrogen receptor modulator (SERM) compound is selected from the group consisting of raloxifene and bazedoxifene.
